(19) United States
(12) Patent Application Publication
(10) Pub. No.: US 2024/0407654 A1
(43) Pub. Date: Dec. 12, 2024

(54) IMPLANTABLE, WIRELESS CARDIAC HEMODYNAMICS MONITOR SYSTEM AND APPLICATIONS OF SAME

(71) Applicant: NORTHWESTERN UNIVERSITY, Evanston, IL (US)

(72) Inventors: John A. Rogers, Wilmette, IL (US); Anthony R. Banks, Evanston, IL (US); Kyeongha Kwon, Evanston, IL (US)

(21) Appl. No.: 18/274,040

(22) PCT Filed: Jan. 31, 2022
(86) PCT No.: PCT/US2022/014534

§ 371 Date: Jul. 25, 2023

Related U.S. Application Data
(60) Provisional application No. 63/143,131, filed on Jan. 29, 2021.

(51) Int. Cl. A61B 5/0205; A61B 5/00; A61B 5/01; A61B 5/021; A61B 5/026

(52) U.S. Cl. CPC A61B 5/02055; A61B 5/6869; A61B 5/6876; A61B 5/01; A61B 5/021; A61B 5/026; A61B 2560/0219; A61B 2560/045; A61B 2560/0462; A61B 2562/0247; A61B 2562/0261; A61B 2562/0271; A61B 2562/227

(57) ABSTRACT

An implantable, wireless cardiac hemodynamics monitor system includes a bio-sensing module and a wireless electronic subsystem. The bio-sensing module is implanted in a heart or an artery of the mammal subject to continuously monitor cardiac functions of the mammal subject. The wireless electronic subsystem is implanted between a fat layer and a dermis layer of a skin of the mammal subject and electrically connected to the bio-sensing module through insulated flexible wires. the wireless electronic subsystem is wirelessly communicated to an external wireless power transfer (WPT) module and an external user interface module. In operation, the wireless electronic subsystem is used to wirelessly receive power transferred from the external WPT module, and provide the power to the bio-sensing module; and to obtain sensing signals of the cardiac functions monitored by the bio-sensing module, and wirelessly transmit the sensing signals obtained to the external user interface module.

### CROSS-REFERENCE TO RELATED APPLICATIONS

This PCT application claims priority to and the benefit of U.S. Provisional Patent Application Ser. No. 63/143,131, which was filed Jan. 29, 2021. The content of the application is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTION

The present invention relates generally to healthcare, and more particularly to an implantable, wireless cardiac hemodynamics monitor system, and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The aortic valve, located between the left ventricle of a heart and the aorta (i.e., the main artery), is an important structure for preventing the back flow of the arterial blood into the heart. The aortic valve has thin leaflets of tissue that open and close when the heart beats to regulate blood flow from the left ventricle (which is the main pumping chamber of the heart) to the main artery (aorta) that supplies oxygen-rich blood to the body. The aortic valve disease is a common pathology in the cardiovascular system which leads to a narrowing of the opening (aortic stenosis) of the valve or to leaking due to incomplete closing (aortic regurgitation). The conventional treatment for severe aortic stenosis in most patients is open-heart surgery with insertion of an artificial valve. Specifically, in selected young adults and children, inflating a balloon attached at the end of a catheter (aortic balloon valvotomy) helps open a stenotic aortic valve.

The gold standard of tracking hemodynamic function is inserting a catheter floated into the artery, and connecting its output port to an external monitor, which displays blood pressure and flow rate in and around the heart. This technology involves a lengthy catheter (e.g. 35 cm for endovascular catheter, Cherry Hill, NJ), a few cm long surface heating element and a high precision thermistor that involve a wired connection to a stationary monitor, thereby limiting its use for temporary, stationary monitoring.

Therefore, a heretofore unaddressed need exists in the art to address the aforementioned deficiencies and inadequacies.

### SUMMARY OF THE INVENTION

One of the objectives of the invention is to provide an implantable, wireless cardiac hemodynamics monitor system, which includes two sub-systems to enable continuous, real-time measurements of blood flow, pressure, and temperature, and to achieve wireless, battery-free operation.

In one aspect, the invention relates to a detection system to monitor cardiac hemodynamics of a mammal subject, which includes: a wearable external monitoring device, comprising an external wireless power transfer (WPT) module and an external user interface module; and an implantable, wireless cardiac hemodynamics monitor system in wireless communication with the external monitoring device, comprising: a bio-sensing module configured to be implanted in a heart or an artery of the mammal subject to continuously monitor cardiac functions of the mammal subject; and a wireless electronic subsystem configured to be implanted between a fat layer and a dermis layer of a skin of the mammal subject and electrically connected to the bio-sensing module through insulated flexible wires, wherein the wireless electronic subsystem is wirelessly communicated to the external monitoring device under a Bluetooth low energy (BLE) communication protocol; wherein the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module.

In certain embodiments, the bio-sensing module is configured to be implanted in the heart of the mammal subject, the wireless electronic subsystem is configured to be implanted in the skin at a chest area of the mammal subject, and the wearable external monitoring device is disposed in a pocket of a vest, such that when the mammal subject wears the vest, the wearable external monitoring device is substantially aligned to the wireless electronic subsystem.

In certain embodiments, the bio-sensing module has a multilayered structure comprising: a substrate; a plurality of sensors disposed on the substrate, configured to measure bi-directional flow rates, pressure and temperature of blood of the mammal subject; a plurality of flexible and stretchable interconnects electrically connecting the sensors; and an elastomeric encapsulation layer at least partially surrounding the substrate, the sensors and the flexible and stretchable interconnects.

In certain embodiments, each of the sensors is formed by a piezoelectric monocrystalline silicon nanomembrane (Si-NM) strain gauge.

In certain embodiments, the sensors include: a flow sensor configured to measure the bi-directional flow rates of the blood; a pressure sensor configured to measure the pressure of the blood; and a temperature sensor configured to measure the temperature of the blood.

In certain embodiments, the flow sensor has a three-dimensional fin structure formed by the Si-NM strain gauge to measure the bi-directional flow of the blood based on tensile and compressive forces to the Si-NM strain gauge caused by the bi-directional flow rate of the blood, and wherein a relationship between a strain e measured by the Si-NM strain gauge to bi-directional flow velocity v of the blood is:

$$ε ( \% ) = {c}_{0} \cdot {v}^{2} + {c}_{1} \cdot v ,$$

wherein c₀ and c₁ are constant coefficients.

In certain embodiments, for the bi-directional flows of the blood, values of c₀ and c₁ are: for a forward flow of the blood, c_{0,f}=0.048 and c_{1,f}=0.0047; and for a back flow of the blood, c_{0,b}=−0.0497 and C_{1,b}=0.0049.

In certain embodiments, the pressure sensor is formed by disposing the Si-NM strain gauge on an air-filled cavity on the substrate to be compressed by the pressure of the blood, and wherein a relationship between a strain e measured by the Si-NM strain gauge to bi-directional flow velocity v of the blood is:

$$ε ( \% ) = c \cdot {[ Δ P \sqrt{S} / {\tilde{E}}_{{SiO}_{2}} {h}_{{SiO}_{2}} + {\tilde{E}}_{Si} {h}_{Si} + {\tilde{E}}_{PI} {h}_{PI} ]}^{\frac{2}{3}} = α \cdot Δ {P}^{\frac{2}{3}} ,$$

wherein c is a constant, S is the surface area, ΔP is the pressure, E is a Young's modulus of the Si-NM strain gauge, and h is a thickness of the Si-NM strain gauge.

In certain embodiments, the wireless electronic subsystem comprises: a plurality of electronic components; a plurality of flexible antenna coils electrically interconnected to the electronic components; and a plurality of bio-compatible encapsulation layers encapsulating the electronic components and the flexible antenna coil.

In certain embodiments, the flexible antenna coils comprises a receiving coil and a transmitting coil, and the electronic components comprise: a power management module electrically connected to the receiving coil, configured to receive and convert the power wirelessly received by the receiving coil, and to provide the power to the bio-sensing module; a Bluetooth low energy (BLE) system on a chip (SoC) electrically connected to the power management module, configured to receive and transmit the power converted by the power management module, and to control the transmitting coil to wirelessly transmit the sensing signals to the external user interface module; and analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

In certain embodiments, the power management module comprises: a bridge rectifier electrically connected to the receiving coil to perform full-wave rectification to the power received by the receiving coil; a charge pump converter electrically connected to the bridge rectifier, configured to regulate voltage of the power received by the receiving coil; and a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

In certain embodiments, the BLE SoC comprises: a plurality of analog to digital converters (ADCs) configured to convert analog signals received by the AFE circuits to the digital data; a general-purpose input/output (GPIO) pin configured to supply the regulated voltage to the AFE circuits; and a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

In certain embodiments, the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

Another aspect of the invention relates to a method of wirelessly monitoring cardiac hemodynamics of a mammal subject, which includes: providing a wearable external monitoring device in a pocket of a vest, wherein the wearable external monitoring device comprises an external wireless power transfer (WPT) module and an external user interface module; implanting a bio-sensing module of an implantable, wireless cardiac hemodynamics monitor system in a heart or an artery of the mammal subject, wherein the bio-sensing module is configured to continuously monitor cardiac functions of the mammal subject; and implanting a wireless electronic subsystem of the implantable, wireless cardiac hemodynamics monitor system between a fat layer and a dermis layer of a skin of the mammal subject, and electrically connecting the wireless electronic subsystem to the bio-sensing module through insulated flexible wires, wherein the wireless electronic subsystem is wirelessly communicated to the external monitoring device under a Bluetooth low energy (BLE) communication protocol, and the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module; wherein when the mammal subject wears the vest, the wearable external monitoring device is substantially aligned to the wireless electronic subsystem.

In certain embodiments, the bio-sensing module has a multilayered structure comprising: a substrate; a plurality of sensors disposed on the substrate, configured to measure bi-directional flow rates, pressure and temperature of blood of the mammal subject; a plurality of flexible and stretchable interconnects electrically connecting the sensors; and an elastomeric encapsulation layer at least partially surrounding the substrate, the sensors and the flexible and stretchable interconnects.

In certain embodiments, each of the sensors is formed by a piezoelectric monocrystalline silicon nanomembrane (Si-NM) strain gauge.

In certain embodiments, the sensors include: a flow sensor configured to measure the bi-directional flow rates of the blood; a pressure sensor configured to measure the pressure of the blood; and a temperature sensor configured to measure the temperature of the blood.

In certain embodiments, the flow sensor has a three-dimensional fin structure formed by the Si-NM strain gauge to measure the bi-directional flow of the blood based on tensile and compressive forces to the Si-NM strain gauge caused by the bi-directional flow rate of the blood, and wherein a relationship between a strain e measured by the Si-NM strain gauge to bi-directional flow velocity v of the blood is:

$$ε ( \% ) = {c}_{0} \cdot {v}^{2} + {c}_{1} \cdot v ,$$

wherein c₀ and c₁ are constant coefficients.

In certain embodiments, for the bi-directional flows of the blood, values of c₀ and c₁ are: for a forward flow of the blood, c_{0,f}=0.048 and c_{1,f}=0.0047; and for a back flow of the blood, c_{0,b}=−0.0497 and C_{1,b}=0.0049.

In certain embodiments, the pressure sensor is formed by disposing the Si-NM strain gauge on an air-filled cavity on the substrate to be compressed by the pressure of the blood, and wherein a relationship between a strain e measured by the Si-NM strain gauge to bi-directional flow velocity v of the blood is:

$$ε ( \% ) = c \cdot {[ Δ P \sqrt{S} / {\tilde{E}}_{{SiO}_{2}} {h}_{{SiO}_{2}} + {\tilde{E}}_{Si} {h}_{Si} + {\tilde{E}}_{PI} {h}_{PI} ]}^{\frac{2}{3}} = α \cdot Δ {P}^{\frac{2}{3}} ,$$

wherein c is a constant, S is the surface area, ΔP is the pressure, E is a Young's modulus of the Si-NM strain gauge, and h is a thickness of the Si-NM strain gauge.

In certain embodiments, the wireless electronic subsystem comprises: a plurality of electronic components; a plurality of flexible antenna coils electrically interconnected to the electronic components; and a plurality of bio-compatible encapsulation layers encapsulating the electronic components and the flexible antenna coil.

In certain embodiments, the flexible antenna coils comprises a receiving coil and a transmitting coil, and the electronic components comprise: a power management module electrically connected to the receiving coil, configured to receive and convert the power wirelessly received by the receiving coil, and to provide the power to the bio-sensing module; a Bluetooth low energy (BLE) system on a chip (SoC) electrically connected to the power management module, configured to receive and transmit the power converted by the power management module, and to control the transmitting coil to wirelessly transmit the sensing signals to the external user interface module; and analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

In certain embodiments, the power management module comprises: a bridge rectifier electrically connected to the receiving coil to perform full-wave rectification to the power received by the receiving coil; a charge pump converter electrically connected to the bridge rectifier, configured to regulate voltage of the power received by the receiving coil; and a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

In certain embodiments, the BLE SoC comprises: a plurality of analog to digital converters (ADCs) configured to convert analog signals received by the AFE circuits to the digital data; a general-purpose input/output (GPIO) pin configured to supply the regulated voltage to the AFE circuits; and a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

In certain embodiments, the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

In yet another aspect of the invention, an implantable, wireless cardiac hemodynamics monitor system includes: a bio-sensing module configured to be implanted in a heart or an artery of a mammal subject to continuously monitor cardiac functions of the mammal subject; and a wireless electronic subsystem configured to be implanted within a skin of the mammal subject and electrically connected to the bio-sensing module through insulated flexible wires, wherein the wireless electronic subsystem is wirelessly communicated to an external wireless power transfer (WPT) module and an external user interface module; wherein the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module.

In certain embodiments, the wireless electronic subsystem is disposed between a fat layer and a dermis layer of the skin of the mammal subject.

In certain embodiments, the wireless electronic subsystem is wirelessly communicated to the external WPT module and the external user interface module bio-sensing module under a Bluetooth low energy (BLE) communication protocol.

In certain embodiments, the external WPT module and the external user interface module collectively form a wearable external monitoring device.

In certain embodiments, the bio-sensing module has a multilayered structure comprising: a substrate; a plurality of sensors disposed on the substrate, configured to measure bi-directional flow rate, pressure and temperature of blood of the mammal subject; a plurality of flexible and stretchable interconnects electrically connecting the sensors; and an elastomeric encapsulation layer at least partially surrounding the substrate, the sensors and the flexible and stretchable interconnects.

In certain embodiments, each of the sensors is formed by a piezoelectric monocrystalline silicon nanomembrane (Si-NM) strain gauge.

In certain embodiments, the sensors comprise: a flow sensor configured to measure the bi-directional flow rates of the blood; a pressure sensor configured to measure the pressure of the blood; and a temperature sensor configured to measure the temperature of the blood.

In certain embodiments, the flow sensor is formed as a three-dimensional fin structure by the Si-NM strain gauge to measure the bi-directional flow of the blood based on tensile and compressive forces to the Si-NM strain gauge caused by the bi-directional flow rate of the blood.

In certain embodiments, a relationship between a strain e measured by the Si-NM strain gauge to bi-directional flow velocity v of the blood is:

$$ε ( \% ) = {c}_{0} \cdot {v}^{2} + {c}_{1} \cdot v ,$$

wherein c₀ and c₁ are constant coefficients.

In certain embodiments, for the bi-directional flows of the blood, values of c₀ and c₁ are: for a forward flow of the blood, c_{0,f}=0.048 and c_{1,f}=0.0047; and for a back flow of the blood, c_{0,b}=−0.0497 and c_{1,b}=0.0049.

In certain embodiments, the pressure sensor is formed by disposing the Si-NM strain gauge on an air-filled cavity on the substrate to be compressed by the pressure of the blood.

In certain embodiments, a relationship between a strain ε measured by the Si-NM strain gauge to bi-directional flow velocity v of the blood is:

$$ε ( \% ) = c \cdot {[ Δ P \sqrt{S} / {\tilde{E}}_{{SiO}_{2}} {h}_{{SiO}_{2}} + {\tilde{E}}_{Si} {h}_{Si} + {\tilde{E}}_{PI} {h}_{PI} ]}^{\frac{2}{3}} = α \cdot Δ {P}^{\frac{2}{3}} ,$$

wherein c is a constant, S is the surface area, ΔP is the pressure, E is a Young's modulus of the Si-NM strain gauge, and h is a thickness of the Si-NM strain gauge.

In certain embodiments, the wireless electronic subsystem comprises: a plurality of electronic components; a plurality of flexible antenna coils electrically interconnected to the electronic components; and a plurality of bio-compatible encapsulation layers encapsulating the electronic components and the flexible antenna coil.

In certain embodiments, the flexible antenna coils comprises a receiving coil and a transmitting coil, and the electronic components comprise: a power management module electrically connected to the receiving coil, configured to receive and convert the power wirelessly received by the receiving coil, and to provide the power to the bio-sensing module; a Bluetooth low energy (BLE) system on a chip (SoC) electrically connected to the power management module, configured to receive and transmit the power converted by the power management module, and to control the transmitting coil to wirelessly transmit the sensing signals to the external user interface module; and analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

In certain embodiments, the power management module comprises: a bridge rectifier electrically connected to the receiving coil to perform full-wave rectification to the power received by the receiving coil; a charge pump converter electrically connected to the bridge rectifier, configured to regulate voltage of the power received by the receiving coil; and a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

In certain embodiments, the BLE SoC comprises: a plurality of analog to digital converters (ADCs) configured to convert analog signals received by the AFE circuits to the digital data; a general-purpose input/output (GPIO) pin configured to supply the regulated voltage to the AFE circuits; and a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

In certain embodiments, the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

Yet a further aspect of the invention relates to a method of wirelessly monitoring cardiac hemodynamics of a mammal subject, which includes: implanting a bio-sensing module in a heart or an artery of the mammal subject, wherein the bio-sensing module is configured to continuously monitor cardiac functions of the mammal subject; implanting a wireless electronic subsystem within a skin of the mammal subject, and electrically connecting the wireless electronic subsystem to the bio-sensing module through insulated flexible wires; and wirelessly communicating the wireless electronic subsystem an external wireless power transfer (WPT) module and an external user interface module, wherein the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module.

In certain embodiments, the wireless electronic subsystem is disposed between a fat layer and a dermis layer of the skin of the mammal subject.

In certain embodiments, the wireless electronic subsystem is wirelessly communicated to the external WPT module and the external user interface module bio-sensing module under a Bluetooth low energy (BLE) communication protocol.

In certain embodiments, the external WPT module and the external user interface module collectively form a wearable external monitoring device.

In certain embodiments, the bio-sensing module has a multilayered structure comprising: a substrate; a plurality of sensors disposed on the substrate, configured to measure bi-directional flow rate, pressure and temperature of blood of the mammal subject; a plurality of flexible and stretchable interconnects electrically connecting the sensors; and an elastomeric encapsulation layer at least partially surrounding the substrate, the sensors and the flexible and stretchable interconnects.

In certain embodiments, each of the sensors is formed by a piezoelectric monocrystalline silicon nanomembrane (Si-NM) strain gauge.

In certain embodiments, the sensors comprise: a flow sensor configured to measure the bi-directional flow rates of the blood; a pressure sensor configured to measure the pressure of the blood; and a temperature sensor configured to measure the temperature of the blood.

In certain embodiments, the flow sensor is formed as a three-dimensional fin structure by the Si-NM strain gauge to measure the bi-directional flow of the blood based on tensile and compressive forces to the Si-NM strain gauge caused by the bi-directional flow rate of the blood.

In certain embodiments, a relationship between a strain ε measured by the Si-NM strain gauge to bi-directional flow velocity v of the blood is:

$$ε ( \% ) = {c}_{0} \cdot {v}^{2} + {c}_{1} \cdot v ,$$

wherein c₀ and c₁ are constant coefficients.

In certain embodiments, for the bi-directional flows of the blood, values of c₀ and c₁ are: for a forward flow of the blood, c_{0,f}=0.048 and c_{1,f}=0.0047; and for a back flow of the blood, c_{0,b}=−0.0497 and C_{1,b}=0.0049.

In certain embodiments, the pressure sensor is formed by disposing the Si-NM strain gauge on an air-filled cavity on the substrate to be compressed by the pressure of the blood.

In certain embodiments, a relationship between a strain ε measured by the Si-NM strain gauge to bi-directional flow velocity v of the blood is:

$$ε ( \% ) = c \cdot {[ Δ P \sqrt{S} / {\tilde{E}}_{{SiO}_{2}} {h}_{{SiO}_{2}} + {\tilde{E}}_{Si} {h}_{Si} + {\tilde{E}}_{PI} {h}_{PI} ]}^{\frac{2}{3}} = α \cdot Δ {P}^{\frac{2}{3}} ,$$

wherein c is a constant, S is the surface area, ΔP is the pressure, E is a Young's modulus of the Si-NM strain gauge, and h is a thickness of the Si-NM strain gauge.

In certain embodiments, the wireless electronic subsystem comprises: a plurality of electronic components; a plurality of flexible antenna coils electrically interconnected to the electronic components; and a plurality of bio-compatible encapsulation layers encapsulating the electronic components and the flexible antenna coil.

In certain embodiments, the flexible antenna coils comprises a receiving coil and a transmitting coil, and the electronic components comprise: a power management module electrically connected to the receiving coil, configured to receive and convert the power wirelessly received by the receiving coil, and to provide the power to the bio-sensing module; a Bluetooth low energy (BLE) system on a chip (SoC) electrically connected to the power management module, configured to receive and transmit the power converted by the power management module, and to control the transmitting coil to wirelessly transmit the sensing signals to the external user interface module; and analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

In certain embodiments, the power management module comprises: a bridge rectifier electrically connected to the receiving coil to perform full-wave rectification to the power received by the receiving coil; a charge pump converter electrically connected to the bridge rectifier, configured to regulate voltage of the power received by the receiving coil; and a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

In certain embodiments, the BLE SoC comprises: a plurality of analog to digital converters (ADCs) configured to convert analog signals received by the AFE circuits to the digital data; a general-purpose input/output (GPIO) pin configured to supply the regulated voltage to the AFE circuits; and a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

In certain embodiments, the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1 shows (A) a schematic view of a heart of a mammal subject, (B) a photo of the heart, and (C) a chart showing pressure related to aortic valve actions according to certain embodiments of the present invention.

FIG. 2 schematically shows (a) a heart being implanted with a cardiac hemodynamics monitor system, (b) an ovine model with the heart being implanted with the cardiac hemodynamics monitor system, (c) the ovine model wearing a vest with a wearable external monitoring device disposed therein, and (d) a bio-sensing module of the cardiac hemodynamics monitor system according to certain embodiments of the present invention.

FIG. 3 schematically shows (a) an exploded view of a bio-sensing module, (b) a flow sensor in the tension and compression states, (c) a pressure sensor in the normal and pressured states, (d) computational predictions for the pressure sensor strains across the full range of physiologically relevant pressures in the aorta and pulmonary artery, (e) FEA results of strain of the flow sensor on the Si-NM as a function of bi-directional flow velocity, and (f) the ΔR/Rₒ to temperature curve of the temperature sensor according to certain embodiments of the present invention.

FIG. 4 shows optimal images of sensors of the bio-sensing module as shown in FIG. 3, including (A) the sensors without the 3D flow sensor on the laser defined silicon structure, (B) the sensors with (bottom) the 3D flow sensor on the laser defined silicon structure, (C) an enlarged view of the temperature sensor (D) an enlarged view of the pressure sensor, and (E) an enlarged view of the flow sensor with a 2D precursor including a strain gauge on a backward-facing cursor according to certain embodiments of the present invention.

FIG. 5 schematically shows a process for manufacturing the pressure and temperature sensors according to certain embodiments of the present invention.

FIG. 6 schematically shows a process for manufacturing the flow sensor according to certain embodiments of the present invention.

FIG. 7 shows the strain distributions on the SI-NM strain gauge for (a) forward flow rates (0.5 m/s, left; 1 m/s, right) and (b) backward flow rates (−0.5 m/s, left; −1 m/s, right), respectively, according to certain embodiment of the present invention.

FIG. 8 shows the mechanical testing setup for the polyimide (PI) substrate of the flow sensor according to certain embodiments of the present invention, where (a) shows a schematic view of the PI (ASTM D638 Type V) prepared for tensile testing, (b) shows an optical image of the test setup for a PI film with a thickness of 12.5 μm, and (c) shows the stress-strain curves of the PI films.

FIG. 9 shows design features and characteristics of a pressure sensor according to certain embodiments of the present invention, where (a) shows a schematic view of a multi-layered pressure sensing device sealing an air-filled cavity (L×W×200 μm-height), (b) shows the FEA results for Si strain (F) variation as a function of parameter used in 2D analytical modeling with different area of the air-filled cavity (W/L=0.48, 0.64, and 1.56, respectively), and (c) shows the slope (a) corresponding sensor sensitivity as a function of W/L (Current design: W/L=0.64). The multilayer membrane from top to bottom contains a PI layer (thickness of 1.5 μm) as a top encapsulation layer against biofluid, a p-doped Si NM strain gauge (area of 380 μm×300 μm and thickness of 200 nm) as a piezoresistive sensing element, and a uniform silicon oxide (SiO₂) layer (thickness of 1 μm) as a bottom encapsulation and sealing layer of the cavity, respectively.

FIG. 10 shows the cross-sectional FEA images on a pressure sensor according to certain embodiments of the present invention, where (a) shows cross-sectional FEA images of the vertical movements of the multilayer membrane, (b) shows corresponding strain distribution under applied pressures of 37.5 mmHg (left), 75 mmHg (middle), and 120 mmHg (right), and (c) shows the multi-layered pressure sensor on air-filled cavity.

FIG. 11 shows resistive changes of a pressure sensor compared to a commercial pressure sensor according to certain embodiments of the present invention, where (a) shows a photograph of an experimental set up to characterize the response of an integrated device (pressure, temperature, flow sensors) to air pressure in syringe, (b) shows the resistance changes of pressure, temperature, flow sensor as a function of applied air pressure ranging from 0 to 160 mmHg, and (c) shows comparison of the response of a pressure sensor to a commercial pressure sensor.

FIG. 12 shows the influence of the implanted 3D structure on the flow according to certain embodiments of the present invention, where (a) shows velocity distribution in the blood vessel, (b) shows pressure distribution in the blood vessel, (c) shows blood flow velocity distribution to the normalized coordinate, and (d) shows the pressure profile distribution to the normalized coordinate. a-b, Velocity (a) and pressure (b) distribution in the blood vessel with a diameter of 23 mm. c-d, Blood flow velocity (c) and pressure (d) profile distribution (x=3.2 LSensor) as a function of the normalized coordinate (y/D). Left panel: far-field velocity v0=0.5 m/s; right panel: far-field velocity v0=1.0 m/s.

FIG. 13 schematically shows (a) a wireless electronic subsystem, (b) an exploded view and a top view of the wireless electronic subsystem, (c) the device being suturing to the animal body, (d) a circuit and block diagram of the wireless electronic subsystem, (e) a simplified WPT module corresponding to the receiving coil of the wireless electronic subsystem, (f) FEA results of the scattering parameter S₂₁ as a function of frequency, (g) FEA results of the scattering parameter S₂₁ as a function of frequency with or without a secondary transmitting coil, (h) the Pe to size Y curve, and (i) the power transmission efficiency (Pₑ=|S₂₁|²×100%), according to certain embodiments of the present invention.

FIG. 14 shows the implantation of the wireless electronic subsystem and the bio-sensing module according to certain embodiments of the present invention, where (a) shows a thin, flexible, battery-free wireless electronic subsystem that subcutaneously inserts between fat and dermis layers to harvest power through a receiver coil (RX coil) resonant at the NFC frequency (13.56 MHz); and (b) shows a mm-scale bio-sensing module (with bi-directional flow, pressure, and temperature sensors; inset) that integrates with a medical stent and implants inside the pulmonary artery (PA) via a minimally invasive transcatheter delivery.

FIG. 15 shows the ex vivo arterial pressure and flow monitoring in the artificial heart systems according to certain embodiments of the present invention, where (a) shows pulmonary arteries of pig, (b) shows implanting the sensor using a surgical clip, and (c) shows the block diagram of the detection system.

FIG. 16 shows three-dimensional protective wings for the flow sensor according to certain embodiments of the present invention, where (a) shows an optical image of the 3D protective wings, and (b) shows FEA results of the strain response of the flow sensor with and without the protective wings.

FIG. 17 shows the sensor implantation process according to certain embodiments of the present invention.

FIG. 18 shows the titled cross-sectional view of the implanted sensor after the insertion and the implantation process of FIG. 17 according to certain embodiments of the present invention.

FIG. 19 shows an optical image of the artificial heart system according to certain embodiments of the present invention.

FIG. 20 shows an optical image of two pairs of copper wires (each pair to be connected with a flow and pressure sensor, respectively) in a polyurethane tube according to certain embodiments of the present invention.

FIG. 21 shows continuous, real-time (200 Hz) data corresponding to (a) pressure and (b) flow rate, respectively, measured from a traditional wired system (P_{com} and f_{com}; black), and from the wireless platform (P_{BLE} and f_{BLE}; blue) over extended (30 s) time according to certain embodiments of the present invention.

FIG. 22 shows the resultant ΔR_{P} (%) response of the pressure sensor according to certain embodiments of the present invention.

FIG. 23 shows the empirical relationship between ΔR_{P} and ΔP (a) and between ΔR_{f} and f (b) determined by FEA according to certain embodiments of the present invention.

FIG. 24 shows the resultant ΔR_{f}(%) response of the flow sensor according to certain embodiments of the present invention.

FIG. 25 shows the integral of ΔTdt corresponding to the area under the thermodilution curve according to certain embodiments of the present invention.

FIG. 26 shows the systolic peak values of LVP_{BLE} and AP_{BLE} (blue) and those of LVP_{com} and AP_{com} (black) according to certain embodiments of the present invention.

FIG. 27 shows the pressure gradient across the AV: (a) normal and (b) diseased aortic valves according to certain embodiments of the present invention.

### DETAILED DESCRIPTION OF THE INVENTION

The present invention will now be described more fully hereinafter with reference to the accompanying drawings, in which exemplary embodiments of the present invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “mammal subject” refers to a living human subject or a living non-human subject. For the purpose of illustration of the invention, the apparatus and method are applied to monitor and/or measure physiological parameters of neonates or infants. It should be appreciated to one skilled in the art that the apparatus can also be applied to monitor and/or measure physiological parameters of children or adults in practice the invention.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

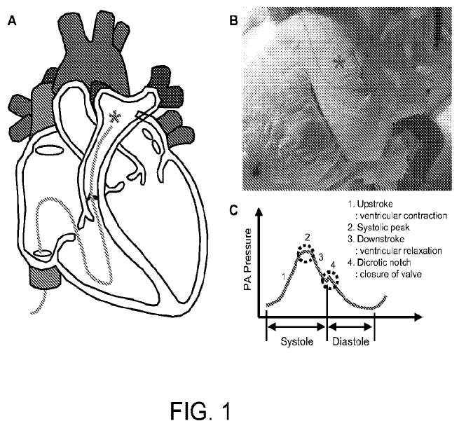

FIG. 1 shows (A) a schematic view of a heart of a mammal subject, (B) a photo of the heart, and (C) a chart showing pressure related to aortic valve actions according to certain embodiments of the present invention. Specifically, as shown in FIGS. 1(A) and (B), the aortic valve has thin leaflets of tissue that open and close when the heart beats to regulate blood flow from the left ventricle to the main artery. As discussed above, the current method for tracking hemodynamic function is inserting a catheter floated into the artery, and connecting its output port to an external monitor, which displays blood pressure and flow rate in and around the heart (e.g., at the area labeled by the star). An example of the pressure being detected is shown in FIG. 1(C). This technology involve a wired connection to a stationary monitor, thereby limiting its use for temporary, stationary monitoring. Thus, emerging techniques such as optical and magnetic flow sensors provide enhanced capabilities for wireless (Bluetooth and NFC) monitoring of blood flow rates.

In one aspect, the invention relates to a detection system to monitor cardiac hemodynamics of a mammal subject, which includes: a wearable external monitoring device, comprising an external wireless power transfer (WPT) module and an external user interface module; and an implantable, wireless cardiac hemodynamics monitor system in wireless communication with the external monitoring device, comprising: a bio-sensing module configured to be implanted in a heart or an artery of the mammal subject to continuously monitor cardiac functions of the mammal subject; and a wireless electronic subsystem configured to be implanted between a fat layer and a dermis layer of a skin of the mammal subject and electrically connected to the bio-sensing module through insulated flexible wires, wherein the wireless electronic subsystem is wirelessly communicated to the external monitoring device under a Bluetooth low energy (BLE) communication protocol; wherein the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module.

Another aspect of the invention relates to a method of wirelessly monitoring cardiac hemodynamics of a mammal subject, which includes: providing a wearable external monitoring device in a pocket of a vest, wherein the wearable external monitoring device comprises an external WPT module and an external user interface module; implanting a bio-sensing module of an implantable, wireless cardiac hemodynamics monitor system in a heart or an artery of the mammal subject, wherein the bio-sensing module is configured to continuously monitor cardiac functions of the mammal subject; and implanting a wireless electronic subsystem of the implantable, wireless cardiac hemodynamics monitor system between a fat layer and a dermis layer of a skin of the mammal subject, and electrically connecting the wireless electronic subsystem to the bio-sensing module through insulated flexible wires, wherein the wireless electronic subsystem is wirelessly communicated to the external monitoring device under a BLE communication protocol, and the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module; wherein when the mammal subject wears the vest, the wearable external monitoring device is substantially aligned to the wireless electronic subsystem.

In yet another aspect of the invention, an implantable, wireless cardiac hemodynamics monitor system includes: a bio-sensing module configured to be implanted in a heart or an artery of a mammal subject to continuously monitor cardiac functions of the mammal subject; and a wireless electronic subsystem configured to be implanted within a skin of the mammal subject and electrically connected to the bio-sensing module through insulated flexible wires, wherein the wireless electronic subsystem is wirelessly communicated to an external WPT module and an external user interface module; wherein the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module.

Yet a further aspect of the invention relates to a method of wirelessly monitoring cardiac hemodynamics of a mammal subject, which includes: implanting a bio-sensing module in a heart or an artery of the mammal subject, wherein the bio-sensing module is configured to continuously monitor cardiac functions of the mammal subject; implanting a wireless electronic subsystem within a skin of the mammal subject, and electrically connecting the wireless electronic subsystem to the bio-sensing module through insulated flexible wires; and wirelessly communicating the wireless electronic subsystem an external WPT module and an external user interface module, wherein the wireless electronic subsystem is configured to: wirelessly receive power transferred from the external WPT module, and provide the power to the bio-sensing module; and obtain sensing signals of the cardiac functions monitored by the bio-sensing module, and wirelessly transmit the sensing signals obtained to the external user interface module.

Specifically, the implantable, wireless cardiac hemodynamics monitor system has two primary parts, including the bio-sensing module and the wireless electronic subsystem. The bio-sensing module has very small sensors that can be implanted in the heart or associated artery. The wireless electronic subsystem functions as a base station, including antenna coils and corresponding control modules that is placed just under the skin on the chest. Micro-sized insulated flexible wires are used to electrically connect the antenna coils and the corresponding control modules to the sensors implanted in the artery or heart. The fully implantable system is wireless powered by an inductively coupled coil in an external monitoring device that is outside the body, and data is transmitted by the base station (i.e., the wireless electronic subsystem) to an external user interface module of the external monitoring device through BLE communication.

In certain embodiments, the small sensors that are implanted in the artery or heart may include different types of sensors that can measure pressure, flow and temperature of blood in real time. This real time measurement can give streaming information about heart/valve operation, blood flow, or arterial health, to name a few as examples.

### Implantable, Wireless Cardiac Hemodynamics Monitor

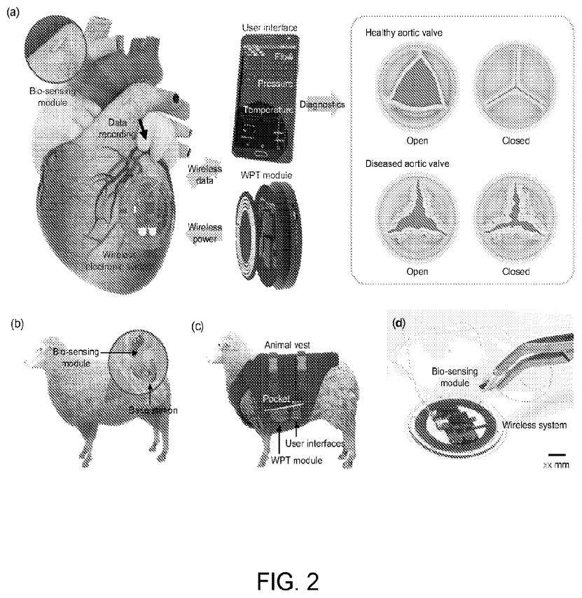

FIG. 2 schematically shows (a) a heart being implanted with a cardiac hemodynamics monitor system, (b) an ovine model with the heart being implanted with the cardiac hemodynamics monitor system, (c) the ovine model wearing a vest with a wearable external monitoring device disposed therein, and (d) a bio-sensing module of the cardiac hemodynamics monitor system according to certain embodiments of the present invention. Specifically, as shown in FIG. 2(a), the cardiac hemodynamics monitor system may include two sub-systems to enable continuous, real-time measurements of blood flow, pressure, and temperature, and to achieve wireless, battery-free operation. Specifically, the bio-sensing module may be an mm-scale bio-sensing module, which is fully implanted inside the arteries (e.g. clipped on the aortic wall) that contains mm-scale bi-directional flow, pressure, and temperature sensors to continuously monitor cardiac function. The wireless electronic subsystem may be a thin, flexible, battery-free wireless subsystem that subcutaneously inserts between the fat layer and the dermis layer of the skin, as shown in FIG. 2(b), to harvest power from the external WPT module of the external monitoring device, and to transmit data to the external user interface (UI) module. The wireless subsystem is electrically connected to the bio-sensing module via insulated, flexible wires, and provides resistive measurements of blood flow, pressure, and temperature via the BLE protocol. The external WPT module may be a portable or a wearable module, which includes an external battery, corresponding charging circuits, and a transmitter coil (TX coil) resonant at a NFC frequency (e.g., 13.56 MHz), and supplies power to the wireless electronic subsystem configured with a receiver coil (RX coil; 13.56 MHz). BLE-enabled UIs (e.g. smartphone, tablet PC) with a customized software control the wireless system, enable storage and analysis of wireless measurements, and display continuous, real-time waveforms of blood flow/pressure/temperature. The external modules, including a WPT module and a UI, are comfortably wearable by putting in the vest pocket, as shown in FIG. 2(c), and positioning the TX coil to be aligned with the RX coil. The integration of both implantable cardiac monitor and external wearables provides critical advantages over other cardiac function measurements in that it provides a technology that is wireless, bio-compatible, miniaturized, portable, user-friendly, and easily accessible. The point-of-care (POC) technology, introduced here, has the potential to allow the rapid detection/characterization of malformations of the heart valves or vessels and malfunctions of prosthetic materials around the heart (e.g. surgical prosthetic valves), and to support continuous and easy cardiac hemodynamics monitoring of individuals or populations peri-, intra-, and post-cardiac surgery. Fabricating and developing such fast and accurate devices can effectively equip cardiac patients or care givers to handle current and probably future heart disease.

### Fully Implantable Bio-Sensing Modules

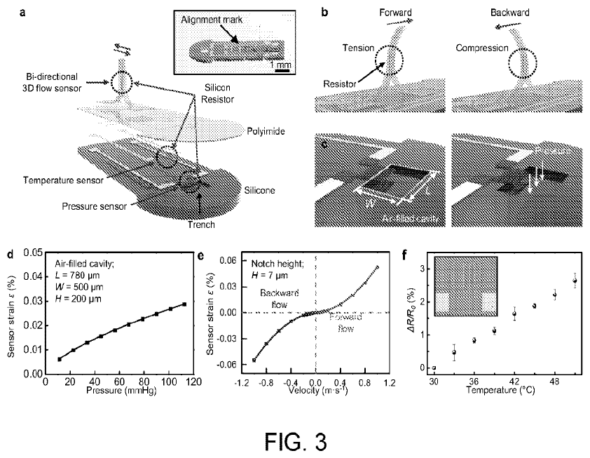

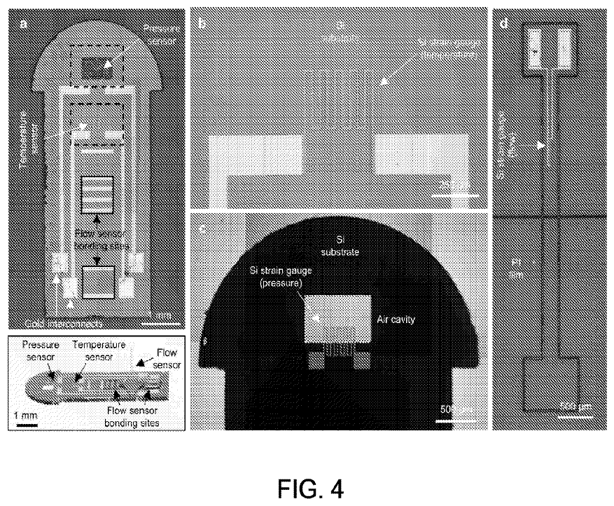

FIG. 3 schematically shows (a) an exploded view of a bio-sensing module, (b) a flow sensor in the tension and compression states, (c) a pressure sensor in the normal and pressured states, (d) computational predictions for the pressure sensor strains across the full range of physiologically relevant pressures in the aorta and pulmonary artery, (e) FEA results of strain of the flow sensor on the Si-NM as a function of bi-directional flow velocity, and (f) the ΔR/Rₒ to temperature curve of the temperature sensor according to certain embodiments of the present invention. FIG. 4 shows optimal images of sensors of the bio-sensing module as shown in FIG. 3, including (A) the sensors without the 3D flow sensor on the laser defined silicon structure, (B) the sensors with (bottom) the 3D flow sensor on the laser defined silicon structure, (C) an enlarged view of the temperature sensor (D) an enlarged view of the pressure sensor, and (E) an enlarged view of the flow sensor with a 2D precursor including a strain gauge on a backward-facing cursor according to certain embodiments of the present invention. Specifically, in certain embodiments, the sensors of the bio-sensing module comprise: a flow sensor configured to measure the bi-directional flow rates of the blood; a pressure sensor configured to measure the pressure of the blood; and a temperature sensor configured to measure the temperature of the blood. For example, as shown in FIG. 3(a), the bio-sensing module incorporates piezoelectric monocrystalline silicon nanomembrane (Si-NM) strain gauges that enable measurements of bi-directional flow rates, pressure, and temperature of the blood in and around the heart. In certain embodiments, the width, length, height, and weight of the device (see inset of FIG. 3(a)) are 3.0 μm, 8.0 μm, 2.3 μm, and 8.0 mg, respectively. In certain embodiments, the bio-sensing module has a multilayered structure comprising: a substrate; a plurality of sensors disposed on the substrate, configured to measure bi-directional flow rates, pressure and temperature of blood of the mammal subject; a plurality of flexible and stretchable interconnects electrically connecting the sensors; and an elastomeric encapsulation layer at least partially surrounding the substrate, the sensors and the flexible and stretchable interconnects. Specifically, as shown in FIG. 3(a), the bio-sensing module has a multilayered structure, including a laser-defined silicon substrate, gold interconnects (see FIGS. 4(a) and (b)), temperature and pressure sensors (see FIGS. 4(c) and (d)) mounted on flat and air-filled parts of the substrate, respectively, a thin (1.5 μm) Polyimide (PI) encapsulation layer, and a bi-directional flow sensor (see FIG. 4(e)) configured with a 3-dimensional (3D) curvy ribbon formed from a 2D precursor, which is a 12.5 m-thick PI film.

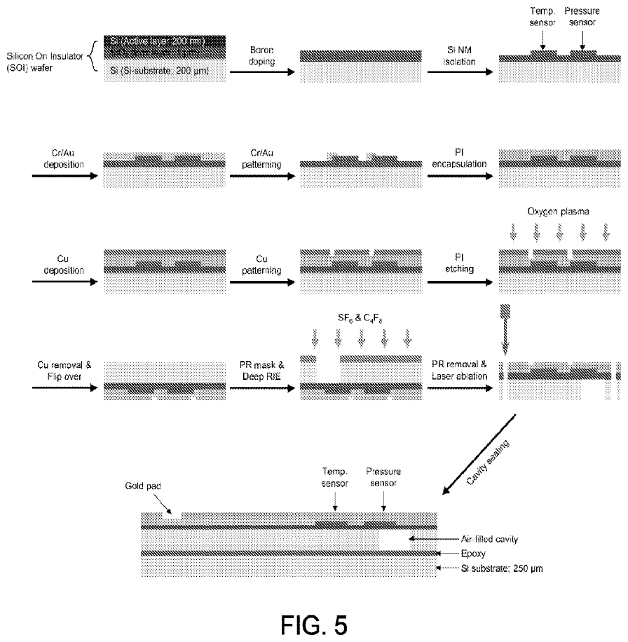

In certain embodiments, the pressure sensor is formed by disposing the Si-NM strain gauge on an air-filled cavity on the substrate to be compressed by the pressure of the blood. FIG. 5 schematically shows a process for manufacturing the pressure and temperature sensors according to certain embodiments of the present invention. As shown in FIG. 5, a silicon on insulator (SOI) wafer is provided, and is then processed through a boron doping process and a Si NM isolation process to form the pressure sensor and the temperature sensor. Then, a series of Cr/Au deposition, Cr/Au patterning, PI encapsulation, Cu deposition, Cu patterning, PI etching and Cu removal processes are performed to form the interconnects and the encapsulating layer. Then, the whole structure is flipped over and performed with the PR mask & deep RIE, PR removal and laser ablation processes to form the air-filled cavities for the pressure sensor. Finally, the cavities are sealed to obtain the whole structure.

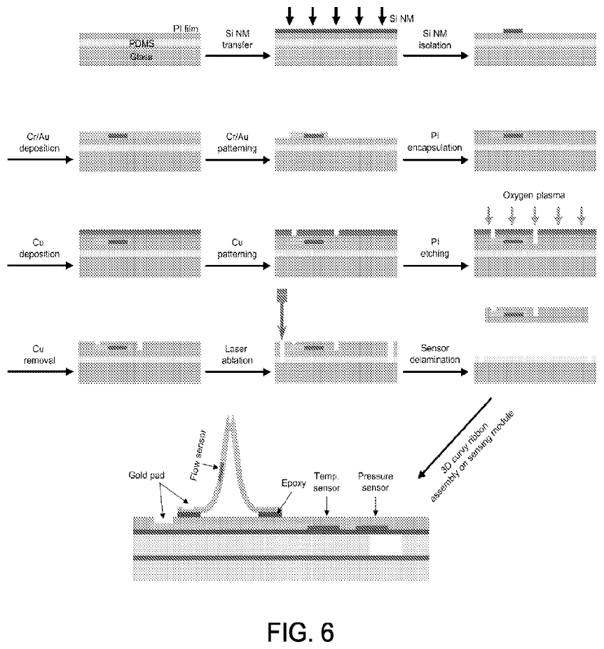

In certain embodiments, the flow sensor has a three-dimensional fin structure formed by the Si-NM strain gauge to measure the bi-directional flow of the blood based on tensile and compressive forces to the Si-NM strain gauge caused by the bi-directional flow rate of the blood. FIG. 6 schematically shows a process for manufacturing the flow sensor according to certain embodiments of the present invention. As shown in FIG. 6, the structure is processed through Si NM transfer and isolation processes to form the Si NM. Then, a series of Cr/Au deposition, Cr/Au patterning, PI encapsulation, Cu deposition, Cu patterning, PI etching and Cu removal processes are performed to form the interconnects and the encapsulating layer. Then, laser ablation and sensor delamination are performed to obtain the 3D curvy ribbon structure. Finally, the 3D curvy ribbon structure is assembled onto the sensing module formed in the process as shown in FIG. 5 to obtain the flow sensor.

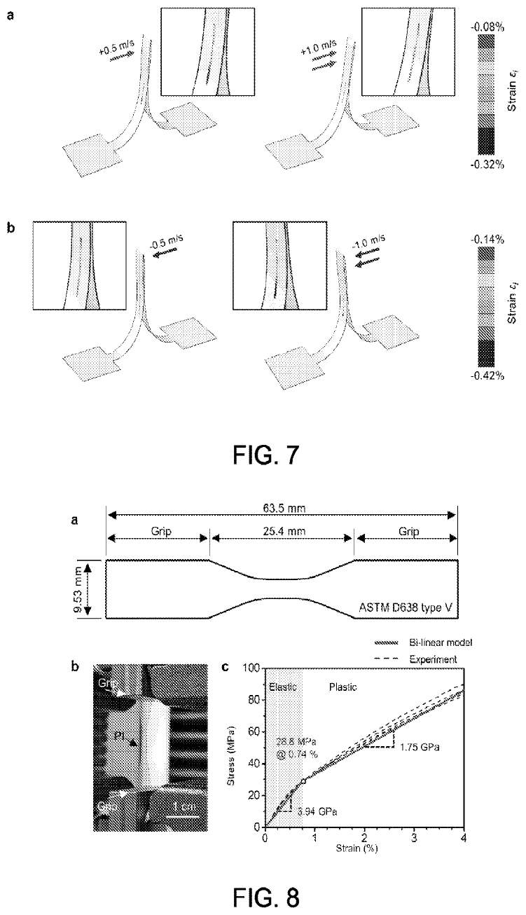

Referring back to FIG. 3(b), the forward and backward flows mechanically deform the 3D fin structure of the flow sensor, which in turn apply tensile (left) and compressive (right) force, respectively, to the backward-facing Si-NM strain gauge and leads a corresponding change of the resistance value. FIG. 7 shows the strain distributions on the SI-NM strain gauge for (a) forward flow rates (0.5 m/s, left; 1 m/s, right) and (b) backward flow rates (−0.5 m/s, left; −1 m/s, right), respectively, according to certain embodiment of the present invention. As shown in FIG. 7, the strain distributions are determined by finite element analysis (FEA). FIG. 3(e) shows FEA results that establish a second-order empirical relationship between the strain response on Si-NM (e) and bi-directional flow velocity (v; m/s):

$$ε ( \% ) = {c}_{0} \cdot {v}^{2} + {c}_{1} \cdot v ,$$

where c_{0,f}=0.048 and c_{1,f}=0.0047 for the forward flow, and c_{0,b}=−0.0497 and c_{1,b}=0.0049 for the backward flow. Details of the FEA will be described later. Here, typical pulmonary (a peak velocity of 0.6-1 m/s) and aortic (mean=0.65 m/s) blood flow in human hearts leads 0.02˜0.05% and −0.02% strains, respectively.

As shown in FIG. 3(c), a pressure sensor includes a Si-NM strain gauge (width, length, and thickness of 380 μm, 300 μm, and 200 nm, respectively) on the top of an air-filled cavity made of a silicon trench. A 1.5 μm-thick PI film as a top encapsulation layer seals the cavity with the width (W), length (L), and height of 500 μm, 780 μm, and 200 μm, respectively (left). The external pressure deforms the membrane to stretch toward the inside the air-filled cavity (right), which leads a consequent change of the resistance value. FIG. 3(d) shows the computational predictions for the sensor strains across the full range of physiologically relevant pressures in the aorta and pulmonary artery. The computational modeling defines the optimized design parameters for the cavity geometry to achieve optimal piezoresistive response stemming from the vertical movement of the pressure sensitive membrane.

FIG. 8 shows the mechanical testing setup for the polyimide (PI) substrate of the flow sensor according to certain embodiments of the present invention, where (a) shows a schematic view of the PI (ASTM D638 Type V) prepared for tensile testing, (b) shows an optical image of the test setup for a PI film with a thickness of 12.5 μm, and (c) shows the stress-strain curves of the PI films. As shown in FIG. 8(c), the elastic strain limit is ˜0.74%.

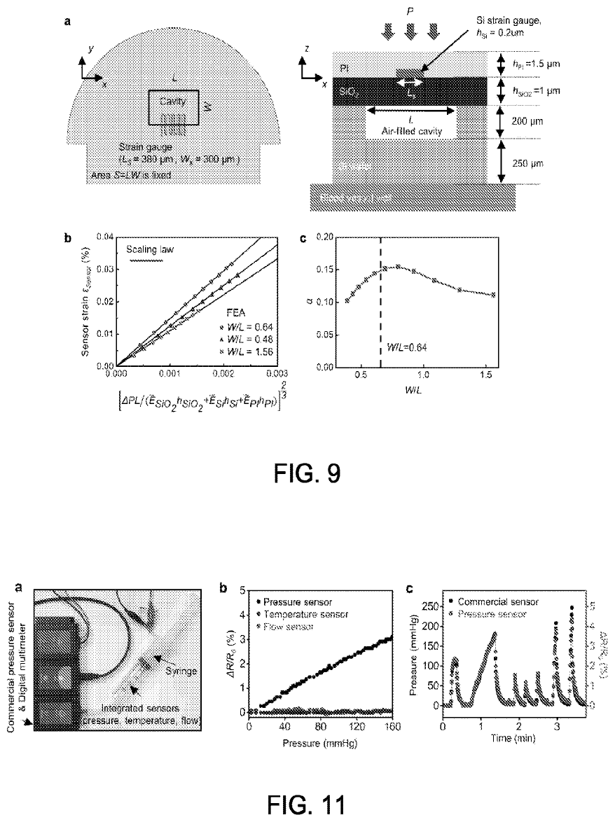

FIG. 9 shows design features and characteristics of a pressure sensor according to certain embodiments of the present invention, where (a) shows a schematic view of a multi-layered pressure sensing device sealing an air-filled cavity (L×W×200 μm-height), (b) shows the FEA results for Si strain (ε) variation as a function of parameter used in 2D analytical modeling with different area of the air-filled cavity (W/L=0.48, 0.64, and 1.56, respectively), and (c) shows the slope (α) corresponding sensor sensitivity as a function of W/L (Current design: W/L=0.64). Specifically, as shown in FIG. 9(a), the multilayer membrane from top to bottom contains a PI layer (thickness of 1.5 μm) as a top encapsulation layer against biofluid, a p-doped Si NM strain gauge (area of 380 μm×300 μm and thickness of 200 nm) as a piezoresistive sensing element, and a uniform silicon oxide (SiO₂) layer (thickness of 1 μm) as a bottom encapsulation and sealing layer of the cavity, respectively. Given the geometrical features of the sensor as shown in FIG. 9(a) and constitutive properties of the materials, an analytical scaling law is given as,

$$ε ( \% ) = c \cdot {[ Δ P \sqrt{S} / {\tilde{E}}_{{SiO}_{2}} {h}_{{SiO}_{2}} + {\tilde{E}}_{Si} {h}_{Si} + {\tilde{E}}_{PI} {h}_{PI} ]}^{\frac{2}{3}} = α \cdot Δ {P}^{\frac{2}{3}}$$

where ε(%) is the strain response on Si-NM, c is a constant, S is the surface area, ΔP is applying pressure, and E and h are Young's modulus and thickness of materials, respectively. As shown in FIGS. 9(b) and 9(c), the slope (α) of a linear empirical relationship between the sensor strain and ΔP^{2/3} corresponds to the sensor sensitivity (e.g. α=0.12, 0.15, and 0.11 for W/L=0.48, 0.80 and 1.56, respectively), which reaches the maximum for W/L=0.80 a fixed surface area of S=0.39 mm² (see FIG. 8(c)).

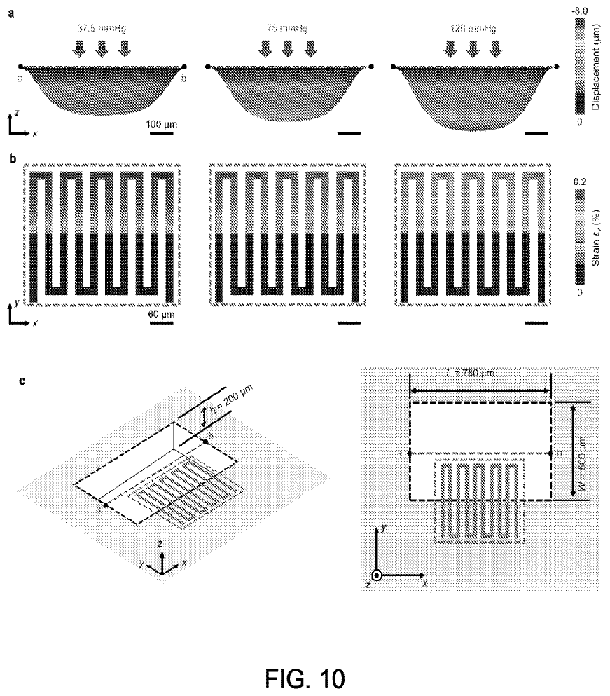

FIG. 10 shows the cross-sectional FEA images on a pressure sensor according to certain embodiments of the present invention, where (a) shows cross-sectional FEA images of the vertical movements of the multilayer membrane, (b) shows corresponding strain distribution under applied pressures of 37.5 mmHg (left), 75 mmHg (middle), and 120 mmHg (right), and (c) shows the multi-layered pressure sensor on air-filled cavity. Specifically, FIGS. 10(a) and (b) presents the cross-sectional (through xz-plane and zy-plane as shown in FIG. 10(c), respectively) FEA images of vertical displacement of the multilayered membrane, and strain distributions, respectively, under applied pressures of 37.5 mmHg (left), 75 mmHg (middle), and 120 mmHg (right).

Referring back to FIG. 3(f), a temperature sensor as shown in the inset of FIG. 3(f) includes a 1.5 μm-thick PI layer as a top encapsulation layer and a Si-NM strain gauge (width, length, and thickness of 380 μm, 300 μm and 200 nm, respectively) on the top of the flat Si substrate. FIG. 11 shows resistive changes of a pressure sensor compared to a commercial pressure sensor according to certain embodiments of the present invention, where (a) shows a photograph of an experimental set up to characterize the response of an integrated device (pressure, temperature, flow sensors) to air pressure in syringe, (b) shows the resistance changes of pressure, temperature, flow sensor as a function of applied air pressure ranging from 0 to 160 mmHg, and (c) shows comparison of the response of a pressure sensor to a commercial pressure sensor. Specifically, the temperature sensor of a fully integrated sensing module located inside a syringe as shown in FIG. 10(a) shows a linear response to temperature changes from 30° C. to 50° C. (˜0.10%/° C. and R²=0.99) as shown in FIG. 3(f) and no response to the change of air pressure inside as shown in FIG. 11(a), whereas the pressure sensor shows the consistent results with commercial pressure sensor, as shown in FIG. 11(c). Implantation of the u-scale bio sensors in aortic and pulmonary arteries affects on flow and pressure distribution and mechanical properties of the arterials walls.

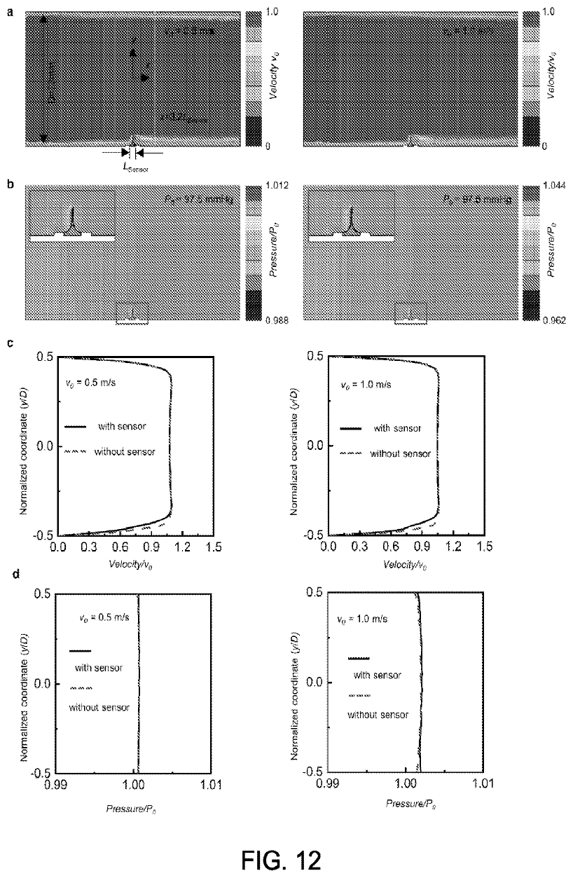

FIG. 12 shows the influence of the implanted 3D structure on the flow according to certain embodiments of the present invention, where (a) shows velocity distribution in the blood vessel, (b) shows pressure distribution in the blood vessel, (c) shows blood flow velocity distribution to the normalized coordinate, and (d) shows the pressure profile distribution to the normalized coordinate. Specifically, for obtaining the data as shown in FIGS. 12(a) and (b), the blood vessel has a diameter of 23 mm. As shown in FIGS. 12(c) and (d), the left panel shows the far-field velocity v₀=0.5 m/s, and the right panel shows the far-field velocity v₀=1.0 m/s.

### Subcutaneous Implants for Wireless Power and Data Transmission

Battery-powered implants limit their operational lifetime, necessitate surgical interventions to replace/recharge the batteries, and pose severe risks from leaks. Wirelessly powered implants are growing in importance as they enable seamless and safe operation, without the needs of batteries. Thus, the wireless electronic subsystem is provided to function as the wirelessly powered implants, and the power can be wirelessly provided to the wireless electronic subsystem by the external WPT module.

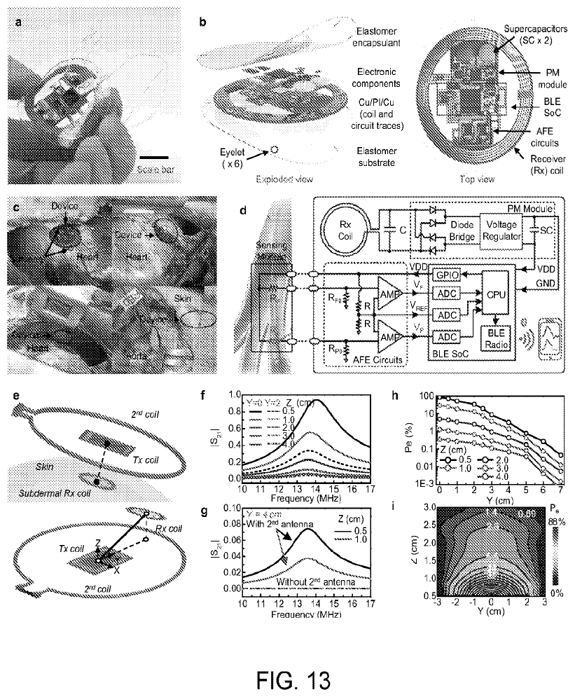

FIG. 13 schematically shows (a) a wireless electronic subsystem, (b) an exploded view and a top view of the wireless electronic subsystem, (c) the device being suturing to the animal body, (d) a circuit and block diagram of the wireless electronic subsystem, (e) a simplified WPT module corresponding to the receiving coil of the wireless electronic subsystem, (f) FEA results of the scattering parameter S₂₁ as a function of frequency, (g) FEA results of the scattering parameter S₂₁ as a function of frequency with or without a secondary transmitting coil, (h) the Pe to size Y curve, and (i) the power transmission efficiency (Pₑ=|S₂₁|²×100%), according to certain embodiments of the present invention. Specifically, as shown in FIG. 13(a), the wireless electronics system achieves three main objectives: (1) wireless power receiving from an external transmitting antenna coil (TX coil), (2) long-range wireless data (blood pressure/flow) transfer to the external user interfaces (e.g. smartphones) and (3) flexible/conformal realizations and tolerance to suit the implantation needs.

In certain embodiments, the wireless electronic subsystem comprises: a plurality of electronic components; a plurality of flexible antenna coils electrically interconnected to the electronic components; and a plurality of bio-compatible encapsulation layers encapsulating the electronic components and the flexible antenna coil. In certain embodiments, the flexible antenna coils comprises a receiving coil and a transmitting coil, and the electronic components comprise: a power management module electrically connected to the receiving coil, configured to receive and convert the power wirelessly received by the receiving coil, and to provide the power to the bio-sensing module; a Bluetooth low energy (BLE) system on a chip (SoC) electrically connected to the power management module, configured to receive and transmit the power converted by the power management module, and to control the transmitting coil to wirelessly transmit the sensing signals to the external user interface module; and analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC. In certain embodiments, the power management module comprises: a bridge rectifier electrically connected to the receiving coil to perform full-wave rectification to the power received by the receiving coil; a charge pump converter electrically connected to the bridge rectifier, configured to regulate voltage of the power received by the receiving coil; and a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil. In certain embodiments, the BLE SoC comprises: a plurality of analog to digital converters (ADCs) configured to convert analog signals received by the AFE circuits to the digital data; a general-purpose input/output (GPIO) pin configured to supply the regulated voltage to the AFE circuits; and a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

Specifically, as shown in FIG. 13(b), the wireless electronic subsystem includes bio-compatible encapsulation layers, off-the-shelf electronic components, and a Cu/PI/Cu sheet processed with a laser cutting tool to yield a thin, flexible antenna coil and circuit traces (left) that interconnect a power management (PM) module, a BLE system on a chip (SoC), and analog front-end (AFE) circuits (right). The thin, flexible encapsulation layer made of silicon elastomer includes multiple eyelets for suturing the platform to the animal body as shown in FIG. 13(c) (specifically, on the left ventricle, left top; under the pericardium, right top; thoracic wall, left bottom; and right under the dermis, right bottom), and provides tolerance to real-time flexion/extension. The circuit and block diagram of the wireless electronics system is shown in FIG. 4(d). In particular, the receiving coil (RX coil) with a capacitor (C=80 pF) resonant at 13.56 MHz allows wireless power receiving from the primary Tx coil (X-NUCLEO-NFC05A1, STMicroelectronics). A bridge rectifier including four diodes provides full-wave rectification from the AC input, and the subsequent charge pump converter regulates the received voltage on the Rx coil to charge a pair of supercapacitors (SCs), and power the BLE SoC and the AFE circuits. The SCs act as a short-term energy buffer during periods with an angular mismatch between TX and RX coils, caused by an unexpected motion of the animal. The remaining circuitry includes the BLE SoC for wireless communication, and the AFE circuits comprising Wheatstone bridge circuits to measure the resistances of bio sensors (R_{F} and R_{P} for flow and pressure sensors, respectively) and the reference voltage (V_{REF}) to monitor the supplied voltage (VDD=2× V_{REF}). The central processing unit (CPU) controls the general-purpose input/output (GPIO) pin to supply a voltage on AFE circuits only at the moment sampling occurs (power profiling), and transmits analog to digital converter (ADC)-sampled data to user interfaces.

In certain embodiments, the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem. Specifically, as shown in FIG. 13(e), the external WPT module, which has a simplified structure, features a main rectangular spiral (width, length, and thickness of 47 mm, 34 mm, and 0.052 mm) TX coil at the center of the secondary circular wire antenna coil (labeled as the 2^{nd} coil; 14.2 cm diameter, 3.4 mm thickness), and transfers wireless power (up to 1.4 W) to a subdermal RX coil of the wireless electronic subsystem implanted (0.5˜ 1 mm) under the skin, as shown in 13(e). The secondary transmitting coil increases the working range of the RX coil placed at a vertical (Z) and horizontal (X, Y) distances from the center of TX coil. FIGS. 13(f) and (g) highlight FEA results that establish the magnitude of the scattering parameter S₂₁ (see FIG. 13(f)) as a function of frequency where the resonance peaks occur at 13.56 MHz, and power transmission efficiency (Pₑ=|S₂₁|²×100%; see FIG. 13(g)) with vertical (Z) and lateral (Y) misalignments. The magnitude of S₂₁ decreases monotonically as the vertical distance Z increases from 0.5 cm to 4 cm (solid and dashed lines for Y=0 cm and 2 cm, respectively). For misalignments in the Y direction of 4 cm as shown in FIG. 13(g) and Z direction of 0.5 cm and 1.0 cm, respectively, the 2^{nd} coil extends the range of wireless power transmission and enables the Rx coil to resonate at 13.56 MHz (solid line), and the Rx coil shows no resonance without the 2^{nd} coil (dashed line). FIG. 13(h) shows the values of Pₑ in logarithmic scale as a function of Y for different Z (from 0.5 cm to 4 cm). The efficiency rapidly decreases with misalignment. FIG. 13(i) shows the color contour with an efficiency of 88% (red color zone) at Z=0.5 cm and Y=0.0 cm, and of 0.69% at Z=3 cm and Y=4 cm.

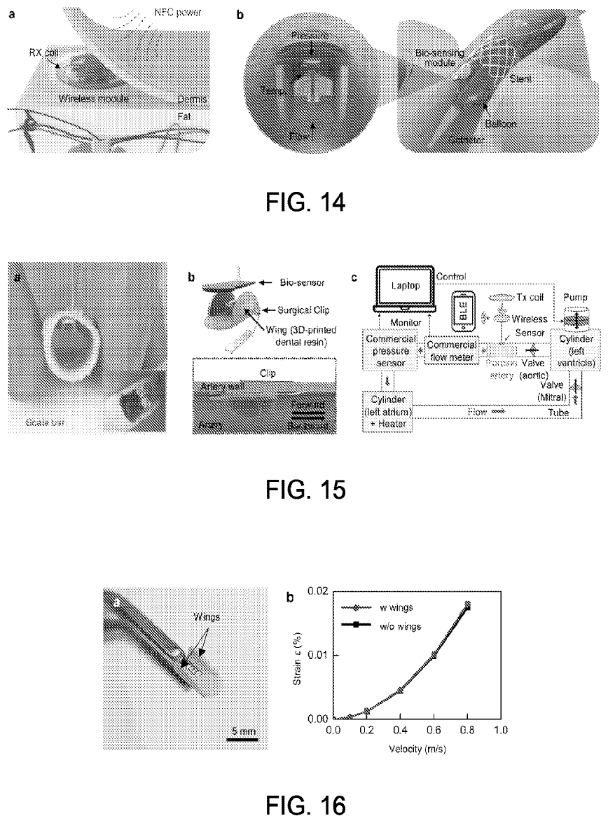

FIG. 14 shows the implantation of the wireless electronic subsystem and the bio-sensing module according to certain embodiments of the present invention, where (a) shows a thin, flexible, battery-free wireless electronic subsystem that subcutaneously inserts between fat and dermis layers to harvest power through a receiver coil (RX coil) resonant at the NFC frequency (13.56 MHz); and (b) shows a mm-scale bio-sensing module (with bi-directional flow, pressure, and temperature sensors; inset) that integrates with a medical stent and implants inside the pulmonary artery (PA) via a minimally invasive transcatheter delivery. This implantation procedure involves an insertion of catheter through the access point, an inflation of a balloon on the catheter's tip to expand the stent into the appropriate position, and removal of the catheter.

The combination of the bio-sensing modules and the wireless electronic subsystem provides continuous blood flow and pressure waveforms, which has the promise to provide clinicians with more clarity and information to deliver fully-wireless, improved care for cardiac patients.

### Examples

The following examples demonstrate the key sensing capabilities and reliability of the measurements for use with large animals (pig and sheep), with comparisons against commercial (flow, pressure sensors) and clinical (swan-ganz catheter) standard devices. Ex vivo and in vivo trials focuses on arterial blood flow and pressure monitoring on pig (pulmonary artery) and sheep (aorta, left ventricle), respectively, which serve as good pre-clinical animal models for heart disease and cardiovascular research.

FIG. 15 shows the ex vivo arterial pressure and flow monitoring in the artificial heart systems according to certain embodiments of the present invention, where (a) shows pulmonary arteries of pig, (b) shows implanting the sensor using a surgical clip, and (c) shows the block diagram of the detection system. Specifically, as shown in FIG. 15(a), the benchtop studies involve the implantation of bio-sensing modules in pulmonary arteries of pig which has similar vasculature, valve structure, and sizing as a human would have, and because of their similarities of organ size, coronary anatomy, immunology, and physiology to humans. In certain embodiments, three-dimensional protective wings may be added to the flow sensor.

The prepared multi-sensing module in blood vessel configures with a surgical clip and 3D-printed wing structures to provide a tight connection between the sensor body and the artery inner wall, and shield the 3D fin structure of a flow sensor during implantation, respectively. As shown in FIG. 15(b), the prepared flow/pressure-sensing module configures a 3D-printed (dental resin) wing structure, and a surgical clip is used to clip the sensor in order to implant the sensor into the artery wall. FIG. 16 shows three-dimensional protective wings for the flow sensor according to certain embodiments of the present invention, where (a) shows an optical image of the 3D protective wings, and (b) shows FEA results of the strain response of the flow sensor with and without the protective wings. Borescope recorded the sensing module inside the artery, and confirmed no flopping under pulsatile flow.

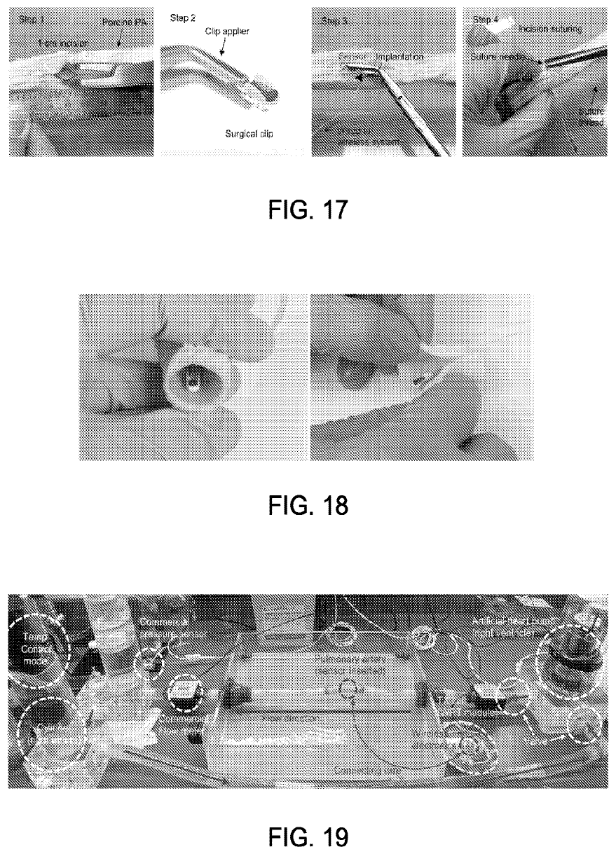

FIG. 17 shows the sensor implantation process according to certain embodiments of the present invention. Specifically, in step 1, a 1-cm incision is formed on the procine PA. In step 2, a surgical clip is used as a clip applier to clip the sensor. In step 3, the sensor is implanted. In step 4, incision suturing is performed using suture needle and threads. FIG. 18 shows the titled cross-sectional view of the implanted sensor after the insertion and the implantation process of FIG. 17 according to certain embodiments of the present invention.

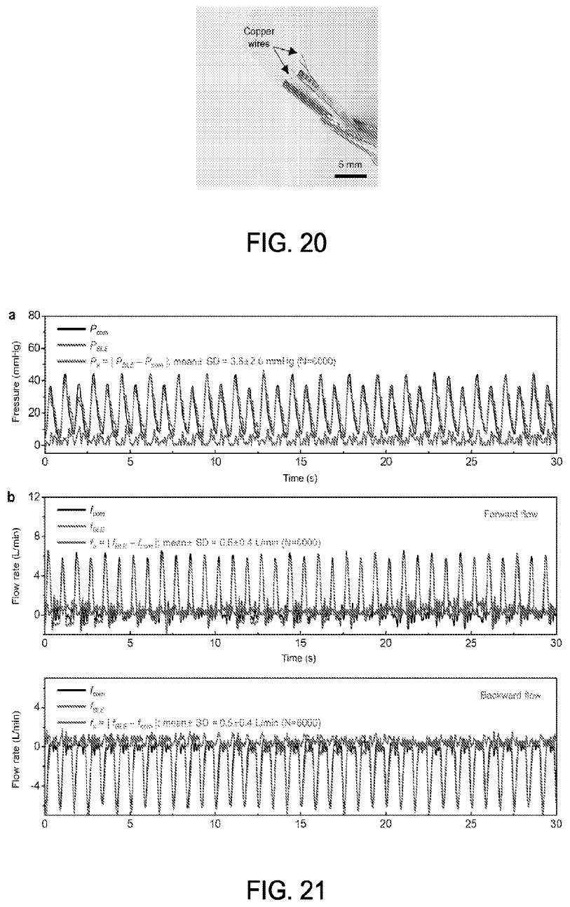

The measurements have been performed on porcine arteries using the detection system according to certain embodiments of the present invention and compared to the commercial and traditional clinical wired devices. Specifically, FIG. 19 shows an optical image of the artificial heart system according to certain embodiments of the present invention. FIG. 20 shows an optical image of two pairs of copper wires (each pair to be connected with a flow and pressure sensor, respectively) in a polyurethane tube according to certain embodiments of the present invention. FIG. 21 shows continuous, real-time (200 Hz) data corresponding to (a) pressure and (b) flow rate, respectively, measured from a traditional wired system (P_{com} and f_{com}; black), and from the wireless platform (P_{BLE} and f_{BLE}; blue) over extended (30 s) time according to certain embodiments of the present invention.

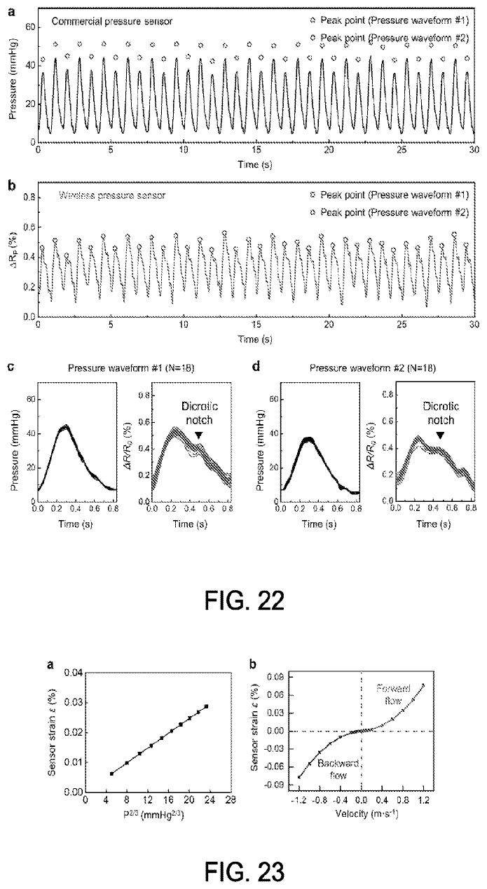

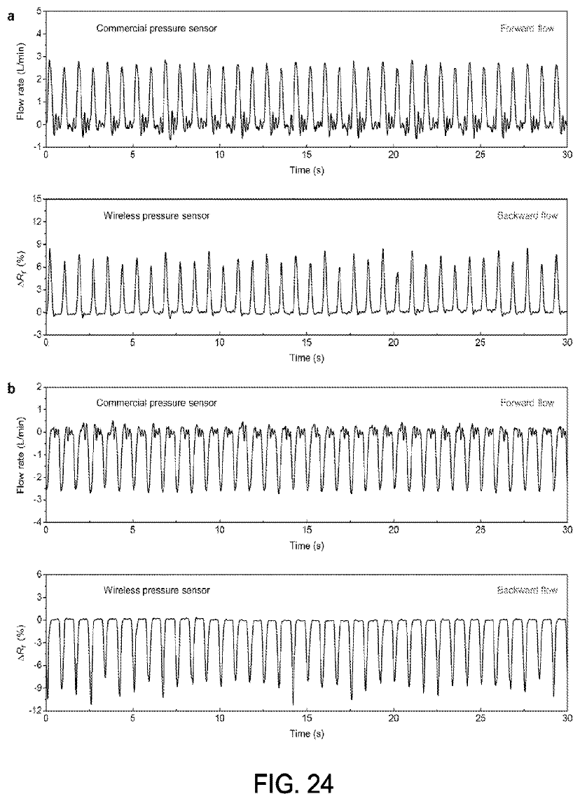

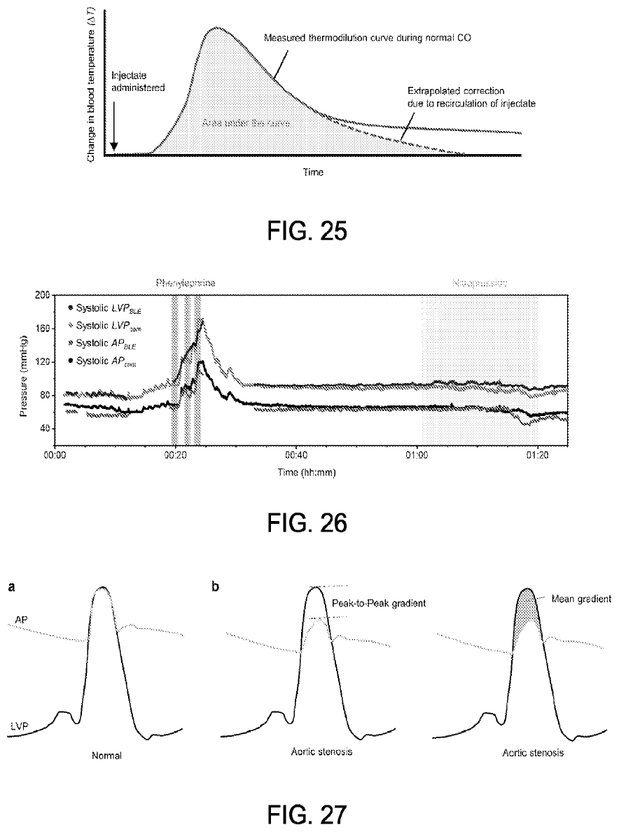

FIG. 22 shows the resultant ΔR_{P} (%) response of the pressure sensor according to certain embodiments of the present invention. FIG. 23 shows the empirical relationship between ΔR_{P} and ΔP (a) and between ΔR_{f} and f(b) determined by FEA according to certain embodiments of the present invention. FIG. 24 shows the resultant ΔR_{f}(%) response of the flow sensor according to certain embodiments of the present invention. FIG. 25 shows the integral of ΔTdt corresponding to the area under the thermodilution curve according to certain embodiments of the present invention. FIG. 26 shows the systolic peak values of LVP_{BLE} and AP_{BLE} (blue) and those of LVP_{com} and AP_{com} (black) according to certain embodiments of the present invention. FIG. 27 shows the pressure gradient across the AV: (a) normal and (b) diseased aortic valves according to certain embodiments of the present invention.

### Finite Element Analysis (FEA)

### 3D Assembly of the Flow Sensor

FEA (commercial software ABAQUS, version 2016) is used to predict the 2D-to-3D shape transformation of the flow sensor induced by the compressive strain. As the crease at the middle of the structure leads to large local strain that may exceed the elastic limit, the polyimide (PI) is modeled by a bi-linear plasticity constitutive model, as shown in the solid line of FIG. 8(c). The uniaxial tension test was performed to obtain the stress-strain curve of PI, as shown in the dashed lines of FIG. 8(c) to fit the material parameters in the constitutive model. The slopes of the first and second stage in the model are E_{PI}=3.94 GPa (Young's modulus) and E_{PI_plastic}=1.75 GPa, respectively, and the yield stress is σ_{yield}=28.8 MPa. Literature value is used for the Poisson's ratio of PI, i.e. v_{PI}=0.34. The silicon and Au traces in the structure are located with a sufficient distance from the crease region of strain concentration, such that the strains in them are well below their elastic limits and therefore modeled by elastic constitutive models, with material properties (Young's modulus E, Poisson's ratio v) E_{Si}=130 GPa, v_{Si}=0.27 for silicon and E_{Au}=78 GPa, v_{Au}=0.44 for gold. Four-node, finite-strain shell elements are used to model the flow sensor.

The Fluid-Structure Interaction of the Flow Sensor with the Blood

To model the deformation of the flow sensor driven by the blood flow, the 3D shape of the buckled flow sensor is imported into the computation fluidic dynamics (CFD) module of ABAQUS as the no-friction, static wall boundary. The blood vessel is modeled as a tube with the diameter and the length much larger that of the flow sensor, such that the blood vessel size does not affect the results. The uniform velocity condition is applied at the inlet to generate the blood flow. The traction forces applied on the flow sensor by the blood flow predicted by the CFD computation is then imported into the mechanics module of ABAQUS to simulate the deformation of the flow sensor. The displacement of the flow sensor is small (<200 μm, give the largest displacement) compared to the sensor size (2.3 mm), which justifies the use of the static wall boundary in the fluid-structure interaction. This sequential coupling process is much more effective than the direct fluid-structure interaction that may encounter convergency issues. The Young's modulus and the Poisson's ratio of the materials involved in this simulation are already presented. The material densities (p) of PI, Si, Au and blood are 1420 kg/m³, 2320 kg/m³, 19300 kg/m³ and 1025 kg/m³ [2, 3], respectively. The blood dynamic viscosity is 0.003 Pa·s. Four-node tetrahedron (FC3D4) fluid elements are used to model the blood and refined mesh around the flow sensor ensures accuracy.

### The Deformation of the Pressure Sensor

A uniform pressure is applied on the top surface of the pressure sensor above the cavity to simulate the deformation, with the bottom surface of the sensor being tration-free. The material properties are E_{SiO}_{₂}=70 GPa, v_{SiO}_{₂}=0.17 for SiO₂, with those for PI and silicon already given. Four-node, finite-strain shell elements are used.

### Scaling Law for the Sensor Strain of the Pressure Sensor

For a cylindrical cavity as shown in FIG. 8, an analytical solution predicts the deformation of the pressure sensor. By neglecting the bending strain, the hoop strain (ε_{θ}) and the radial strain (εᵣ) can be obtained as

$${ε}_{θ} = {ε}_{r} = \frac{1}{6} {π}^{\frac{1}{3}} \cdot {[ Δ P \sqrt{S} / ( {\tilde{E}}_{{SiO}_{2}} {h}_{{SiO}_{2}} + {\tilde{E}}_{Si} {h}_{Si} + {\tilde{E}}_{PI} {h}_{PI} ) ]}^{\frac{2}{3}} = α \cdot Δ {P}^{\frac{2}{3}}$$

where S is the cross-sectional area of the cavity, \tilde{E}_{SiO}_{₂}h_{SiO}_{₂}+\tilde{E}_{Si}h_{Si}+\tilde{E}_{PI}h_{PI} is the tensile rigidity of the pressure sensor, with \tilde{E}_{SiO}_{₂}=E_{SiO}_{₂}/(1−v_{SiO}_{₂}), \tilde{E}_{Si}=E_{Si}/(1−v_{Si}) and \tilde{E}_{PI}=E_{PI}/(1−v_{PI}). For the cavity with rectangular cross section, FEA shows that the sensor strain is still linearly proportional to the parameter combination [ΔP\sqrt{S}/(\tilde{E}_{SiO}_{₂}h_{SiO}_{₂}+\tilde{E}_{Si}h_{Si}+\tilde{E}_{PI}h_{PI})]^{2/3}, except that the slope is related with the aspect ratio of the rectangular cross section.

### Pressure Gradient (ΔP) Between LV and Aorta by Aortic Valve Stenosis

For blood to flow from the heart to the aorta, a difference in blood pressure (i.e. pressure gradient (ΔP=LVP-AP)) must exist across the aortic valve. Laminar flow in Newtonian fluids with constant viscosity is governed by the Hagen-Poiseuille equation,

$$F = \frac{Δ P}{R} = Δ P \cdot \frac{π {r}^{4}}{8 η L}$$

where F is volume flow rate, ΔP is pressure gradient, R is resistance to flow, r is radius of tubing, η is fluid viscosity, and L is length of tubing, respectively. In other words, the ΔP is proportional to F and R as well as inversely proportional to the fourth power of the r. Although blood is a non-Newtonian fluid, the Poiseuille relationship distinctly shows the dominant effect of vascular radius (r) on resistance and flow. Therefore, small changes in radius (r) stemming from aortic valve stenosis leads to increasing resistance (R) of flow and ultimately increases in AP.

The foregoing description of the exemplary embodiments of the present invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

1. Nishimura, R. A. Aortic valve disease. Circulation 106, 770-772 (2002).
2. Sawada, R., Nogami, H., Inoue, R. & Higurashi, E. Blood flow sensor with built-in contact pressure and temperature sensor. Symp. Des. Test, Integr. Packag. MEMS/MOEMS, DTIP 2018 1-4 (2018) doi:10.1109/DTIP.2018.8394232.
3. Inoue, R., Nogami, H., Higurashi, E. & Sawada, R. Simultaneous measurement of a blood flow and a contact pressure. BIOSIGNALS 2018-11th Int. Conf Bio-Inspired Syst. Signal Process. Proceedings; Part 11th Int. Jt. Conf Biomed. Eng. Syst. Technol. BIOSTEC 2018 4, 48-53 (2018).
4. Ruiz-Vargas, A., Morris, S. A., Hartley, R. H. & Arkwright, J. W. Optical flow sensor for continuous invasive measurement of blood flow velocity. J Biophotonics 12, 1-8 (2019).
5. Vennemann, B., Obrist, D. & Ro, T. A smartphone-enabled wireless and batteryless implantable blood flow sensor for remote monitoring of prosthetic heart valve function. 1-20 (2020) doi:10.1371/journal.pone.0227372.
6. Laver, R. D., Wiersema, U. F. & Bersten, A. D. Echocardiographic estimation of mean pulmonary artery pressure in critically ill patients. Crit. Ultrasound J. 6, 1-7 (2014).
7. Garcia, J. et al. Distribution of Blood Flow Velocity in the Normal Aorta: Effect of Age and Gender. HHS Public Access 47, 487-498 (2019).
8. Hearse, D. J. & Sutherland, F. J. Experimental Models for the Study of Cardiovascular Function and Disease Defining the Question and Identifying the Model. Pharmacol. Res. 41, (2000).
9. Lukics, E. et al. Overview of large animal myocardial infarction models (review). Acta Physiol. Hung. 99, 365-381 (2012).
10. Milani-Nejad, N. & Janssen, P. M. L. Small and large animal models in cardiac contraction research: Advantages and disadvantages. Pharmacol. Ther. 141, 235-249 (2014).
11. Camacho, P., Fan, H., Liu, Z. & He, J. Q. Small mammalian animal models of heart disease. Am. J. Cardiovasc. Dis. 6, 70-80 (2016).
[S1] Bauer, C. L. and Farris, R. J. (1989). Determination of Poisson's ratio for polyimide films. Polymer Engineering & Science, 29(16), 1107-1110.
[S2] Benson, Katherine. MCAT Review. Emory University. 1999.
[S3] Kwaan, H. (2010). Role of plasma proteins in whole blood viscosity: A brief clinical review. Clinical Hemorheology and Microcirculation, 44(3), 167-176

What is claimed is:

1. A detection system to monitor cardiac hemodynamics of a mammal subject, comprising:
a wearable external monitoring device, comprising an external wireless power transfer (WPT) module and an external user interface module; and
an implantable, wireless cardiac hemodynamics monitor system in wireless communication with the external monitoring device, comprising:
a bio-sensing module configured to be implanted in a heart or an artery of the mammal subject to continuously monitor cardiac functions of the mammal subject; and
a wireless electronic subsystem configured to be implanted between a fat layer and a dermis layer of a skin of the mammal subject and electrically connected to the bio-sensing module through insulated flexible wires, wherein the wireless electronic subsystem is wirelessly communicated to the external monitoring device under a Bluetooth low energy (BLE) communication protocol;
wherein the wireless electronic subsystem is configured to:
wirelessly receive power transferred from the external WPT module, and provide the power to the bio-sensing module; and
obtain sensing signals of the cardiac functions monitored by the bio-sensing module, and wirelessly transmit the sensing signals obtained to the external user interface module.

2. The detection system of claim 1, wherein the bio-sensing module is configured to be implanted in the heart of the mammal subject, the wireless electronic subsystem is configured to be implanted in the skin at a chest area of the mammal subject, and the wearable external monitoring device is disposed in a pocket of a vest, such that when the mammal subject wears the vest, the wearable external monitoring device is substantially aligned to the wireless electronic subsystem.

3. The detection system of claim 1, wherein the bio-sensing module has a multilayered structure comprising:
a substrate;
a plurality of sensors disposed on the substrate, configured to measure bi-directional flow rates, pressure and temperature of blood of the mammal subject;
a plurality of flexible and stretchable interconnects electrically connecting the sensors; and
an elastomeric encapsulation layer at least partially surrounding the substrate, the sensors and the flexible and stretchable interconnects.

4. The detection system of claim 3, wherein each of the sensors is formed by a piezoelectric monocrystalline silicon nanomembrane (Si-NM) strain gauge.

5. The detection system of claim 4, wherein the sensors comprise:
a flow sensor configured to measure the bi-directional flow rates of the blood;
a pressure sensor configured to measure the pressure of the blood; and
a temperature sensor configured to measure the temperature of the blood.

6. The detection system of claim 5, wherein the flow sensor has a three-dimensional fin structure formed by the Si-NM strain gauge to measure the bi-directional flow of the blood based on tensile and compressive forces to the Si-NM strain gauge caused by the bi-directional flow rate of the blood, and wherein a relationship between a strain ε measured by the Si-NM strain gauge to bi-directional flow velocity v of the blood is:
$$ε ( \% ) = {c}_{0} \cdot {v}^{2} + {c}_{1} \cdot v ,$$
wherein c₀ and c₁ are constant coefficients.

7. The detection system of claim 6, wherein for the bi-directional flows of the blood, values of c₀ and c₁ are:
for a forward flow of the blood, c_{0,f}=0.048 and c_{1,f}=0.0047; and
for a back flow of the blood, c_{0,b}=−0.0497 and c_{1,b}=0.0049.

8. The detection system of claim 5, wherein the pressure sensor is formed by disposing the Si-NM strain gauge on an air-filled cavity on the substrate to be compressed by the pressure of the blood, and wherein a relationship between a strain ε measured by the Si-NM strain gauge to bi-directional flow velocity v of the blood is:
$$ε ( \% ) = c \cdot {[ Δ P \sqrt{S} / {\tilde{E}}_{S i {O}_{2}} {h}_{S i {O}_{2}} + {\tilde{E}}_{S i} {h}_{S i} + {\tilde{E}}_{P I} {h}_{PI} ]}^{\frac{2}{3}} = α \cdot Δ {P}^{\frac{2}{3}} ,$$
wherein c is a constant, S is the surface area, ΔP is the pressure, E is a Young's modulus of the Si-NM strain gauge, and h is a thickness of the Si-NM strain gauge.

9. The detection system of claim 1, wherein the wireless electronic subsystem comprises:
a plurality of electronic components;
a plurality of flexible antenna coils electrically interconnected to the electronic components; and
a plurality of bio-compatible encapsulation layers encapsulating the electronic components and the flexible antenna coil.

10. The detection system of claim 9, wherein the flexible antenna coils comprises a receiving coil and a transmitting coil, and the electronic components comprise:
a power management module electrically connected to the receiving coil, configured to receive and convert the power wirelessly received by the receiving coil, and to provide the power to the bio-sensing module;
a Bluetooth low energy (BLE) system on a chip (SoC) electrically connected to the power management module, configured to receive and transmit the power converted by the power management module, and to control the transmitting coil to wirelessly transmit the sensing signals to the external user interface module; and
analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

11. The detection system of claim 10, wherein the power management module comprises:
a bridge rectifier electrically connected to the receiving coil to perform full-wave rectification to the power received by the receiving coil;
a charge pump converter electrically connected to the bridge rectifier, configured to regulate voltage of the power received by the receiving coil; and
a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

12. The detection system of claim 11, wherein the BLE SoC comprises:
a plurality of analog to digital converters (ADCs) configured to convert analog signals received by the AFE circuits to the digital data;
a general-purpose input/output (GPIO) pin configured to supply the regulated voltage to the AFE circuits; and
a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

13. The detection system of claim 10, wherein the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

14. A method of wirelessly monitoring cardiac hemodynamics of a mammal subject, comprising:
providing a wearable external monitoring device in a pocket of a vest, wherein the wearable external monitoring device comprises an external wireless power transfer (WPT) module and an external user interface module;
implanting a bio-sensing module of an implantable, wireless cardiac hemodynamics monitor system in a heart or an artery of the mammal subject, wherein the bio-sensing module is configured to continuously monitor cardiac functions of the mammal subject; and
implanting a wireless electronic subsystem of the implantable, wireless cardiac hemodynamics monitor system between a fat layer and a dermis layer of a skin of the mammal subject, and electrically connecting the wireless electronic subsystem to the bio-sensing module through insulated flexible wires, wherein the wireless electronic subsystem is wirelessly communicated to the external monitoring device under a Bluetooth low energy (BLE) communication protocol, and the wireless electronic subsystem is configured to:
wirelessly receive power transferred from the external WPT module, and provide the power to the bio-sensing module; and
obtain sensing signals of the cardiac functions monitored by the bio-sensing module, and wirelessly transmit the sensing signals obtained to the external user interface module;
wherein when the mammal subject wears the vest, the wearable external monitoring device is substantially aligned to the wireless electronic subsystem.

15. The method of claim 14, wherein the bio-sensing module has a multilayered structure comprising:
a substrate;
a plurality of sensors disposed on the substrate, configured to measure bi-directional flow rates, pressure and temperature of blood of the mammal subject;
a plurality of flexible and stretchable interconnects electrically connecting the sensors; and
an elastomeric encapsulation layer at least partially surrounding the substrate, the sensors and the flexible and stretchable interconnects.

16. The method of claim 15, wherein each of the sensors is formed by a piezoelectric monocrystalline silicon nanomembrane (Si-NM) strain gauge.

17. The method of claim 16, wherein the sensors comprise:
a flow sensor configured to measure the bi-directional flow rates of the blood;
a pressure sensor configured to measure the pressure of the blood; and
a temperature sensor configured to measure the temperature of the blood.

18. The method of claim 17, wherein the flow sensor has a three-dimensional fin structure formed by the Si-NM strain gauge to measure the bi-directional flow of the blood based on tensile and compressive forces to the Si-NM strain gauge caused by the bi-directional flow rate of the blood, and wherein a relationship between a strain ε measured by the Si-NM strain gauge to bi-directional flow velocity v of the blood is:
$$ε ( \% ) = {c}_{0} \cdot {v}^{2} + {c}_{1} \cdot v ,$$
wherein c₀ and c₁ are constant coefficients.

19. The method of claim 18, wherein for the bi-directional flows of the blood, values of c₀ and c₁ are:
for a forward flow of the blood, c_{0,f}=0.048 and c_{1,f}=0.0047; and
for a back flow of the blood, c_{0,b}=−0.0497 and c_{1,b}=0.0049.

20. The method of claim 17, wherein the pressure sensor is formed by disposing the Si-NM strain gauge on an air-filled cavity on the substrate to be compressed by the pressure of the blood, and wherein a relationship between a strain ε measured by the Si-NM strain gauge to bi-directional flow velocity v of the blood is:
$$ε ( \% ) = c \cdot {[ Δ P \sqrt{S} / {\tilde{E}}_{S i {O}_{2}} {h}_{S i {O}_{2}} + {\tilde{E}}_{S i} {h}_{S i} + {\tilde{E}}_{P I} {h}_{PI} ]}^{\frac{2}{3}} = α \cdot Δ {P}^{\frac{2}{3}} ,$$
wherein c is a constant, S is the surface area, ΔP is the pressure, E is a Young's modulus of the Si-NM strain gauge, and h is a thickness of the Si-NM strain gauge.

21. The method of claim 13, wherein the wireless electronic subsystem comprises:
a plurality of electronic components;
a plurality of flexible antenna coils electrically interconnected to the electronic components; and
a plurality of bio-compatible encapsulation layers encapsulating the electronic components and the flexible antenna coil.

22. The method of claim 21, wherein the flexible antenna coils comprises a receiving coil and a transmitting coil, and the electronic components comprise:
a power management module electrically connected to the receiving coil, configured to receive and convert the power wirelessly received by the receiving coil, and to provide the power to the bio-sensing module;
a Bluetooth low energy (BLE) system on a chip (SoC) electrically connected to the power management module, configured to receive and transmit the power converted by the power management module, and to control the transmitting coil to wirelessly transmit the sensing signals to the external user interface module; and
analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

23. The method of claim 22, wherein the power management module comprises:
a bridge rectifier electrically connected to the receiving coil to perform full-wave rectification to the power received by the receiving coil;
a charge pump converter electrically connected to the bridge rectifier, configured to regulate voltage of the power received by the receiving coil; and
a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

24. The method of claim 23, wherein the BLE SoC comprises:
a plurality of analog to digital converters (ADCs) configured to convert analog signals received by the AFE circuits to the digital data;
a general-purpose input/output (GPIO) pin configured to supply the regulated voltage to the AFE circuits; and
a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

25. The method of claim 22, wherein the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

26. An implantable, wireless cardiac hemodynamics monitor system, comprising:
a bio-sensing module configured to be implanted in a heart or an artery of a mammal subject to continuously monitor cardiac functions of the mammal subject; and
a wireless electronic subsystem configured to be implanted within a skin of the mammal subject and electrically connected to the bio-sensing module through insulated flexible wires, wherein the wireless electronic subsystem is wirelessly communicated to an external wireless power transfer (WPT) module and an external user interface module;
wherein the wireless electronic subsystem is configured to:
wirelessly receive power transferred from the external WPT module, and provide the power to the bio-sensing module; and
obtain sensing signals of the cardiac functions monitored by the bio-sensing module, and wirelessly transmit the sensing signals obtained to the external user interface module.

27. The implantable, wireless cardiac hemodynamics monitor system of claim 26, wherein the wireless electronic subsystem is disposed between a fat layer and a dermis layer of the skin of the mammal subject.

28. The implantable, wireless cardiac hemodynamics monitor system of claim 26, wherein the wireless electronic subsystem is wirelessly communicated to the external WPT module and the external user interface module bio-sensing module under a Bluetooth low energy (BLE) communication protocol.

29. The implantable, wireless cardiac hemodynamics monitor system of claim 26, wherein the external WPT module and the external user interface module collectively form a wearable external monitoring device.

30. The implantable, wireless cardiac hemodynamics monitor system of claim 26, wherein the bio-sensing module has a multilayered structure comprising:
a substrate;
a plurality of sensors disposed on the substrate, configured to measure bi-directional flow rate, pressure and temperature of blood of the mammal subject;
a plurality of flexible and stretchable interconnects electrically connecting the sensors; and
an elastomeric encapsulation layer at least partially surrounding the substrate, the sensors and the flexible and stretchable interconnects.

31. The implantable, wireless cardiac hemodynamics monitor system of claim 30, wherein each of the sensors is formed by a piezoelectric monocrystalline silicon nanomembrane (Si-NM) strain gauge.

32. The implantable, wireless cardiac hemodynamics monitor system of claim 31, wherein the sensors comprise:
a flow sensor configured to measure the bi-directional flow rates of the blood;
a pressure sensor configured to measure the pressure of the blood; and
a temperature sensor configured to measure the temperature of the blood.

33. The implantable, wireless cardiac hemodynamics monitor system of claim 32, wherein the flow sensor is formed as a three-dimensional fin structure by the Si-NM strain gauge to measure the bi-directional flow of the blood based on tensile and compressive forces to the Si-NM strain gauge caused by the bi-directional flow rate of the blood.

34. The implantable, wireless cardiac hemodynamics monitor system of claim 33, wherein a relationship between a strain ε measured by the Si-NM strain gauge to bi-directional flow velocity v of the blood is:
$$ε ( \% ) = {c}_{0} \cdot {v}^{2} + {c}_{1} \cdot v ,$$
wherein c₀ and c₁ are constant coefficients.

35. The implantable, wireless cardiac hemodynamics monitor system of claim 34, wherein for the bi-directional flows of the blood, values of c₀ and c₁ are:
for a forward flow of the blood, c_{0,f}=0.048 and c_{1,f}=0.0047; and
for a back flow of the blood, c_{0,b}=−0.0497 and c_{1,b}=0.0049.

36. The implantable, wireless cardiac hemodynamics monitor system of claim 32, wherein the pressure sensor is formed by disposing the Si-NM strain gauge on an air-filled cavity on the substrate to be compressed by the pressure of the blood.

37. The implantable, wireless cardiac hemodynamics monitor system of claim 36, wherein a relationship between a strain ε measured by the Si-NM strain gauge to bi-directional flow velocity v of the blood is:
$$ε ( \% ) = c \cdot {[ Δ P \sqrt{S} / {\tilde{E}}_{S i {O}_{2}} {h}_{S i {O}_{2}} + {\tilde{E}}_{S i} {h}_{S i} + {\tilde{E}}_{P I} {h}_{PI} ]}^{\frac{2}{3}} = α \cdot Δ {P}^{\frac{2}{3}} ,$$
wherein c is a constant, S is the surface area, ΔP is the pressure, E is a Young's modulus of the Si-NM strain gauge, and h is a thickness of the Si-NM strain gauge.

38. The implantable, wireless cardiac hemodynamics monitor system of claim 26, wherein the wireless electronic subsystem comprises:
a plurality of electronic components;
a plurality of flexible antenna coils electrically interconnected to the electronic components; and
a plurality of bio-compatible encapsulation layers encapsulating the electronic components and the flexible antenna coil.

39. The implantable, wireless cardiac hemodynamics monitor system of claim 38, wherein the flexible antenna coils comprises a receiving coil and a transmitting coil, and the electronic components comprise:
a power management module electrically connected to the receiving coil, configured to receive and convert the power wirelessly received by the receiving coil, and to provide the power to the bio-sensing module;
a Bluetooth low energy (BLE) system on a chip (SoC) electrically connected to the power management module, configured to receive and transmit the power converted by the power management module, and to control the transmitting coil to wirelessly transmit the sensing signals to the external user interface module; and
analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

40. The implantable, wireless cardiac hemodynamics monitor system of claim 39, wherein the power management module comprises:
a bridge rectifier electrically connected to the receiving coil to perform full-wave rectification to the power received by the receiving coil;
a charge pump converter electrically connected to the bridge rectifier, configured to regulate voltage of the power received by the receiving coil; and
a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

41. The implantable, wireless cardiac hemodynamics monitor system of claim 40, wherein the BLE SoC comprises:
a plurality of analog to digital converters (ADCs) configured to convert analog signals received by the AFE circuits to the digital data;
a general-purpose input/output (GPIO) pin configured to supply the regulated voltage to the AFE circuits; and
a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

42. The implantable, wireless cardiac hemodynamics monitor system of claim 39, wherein the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.

43. A method of wirelessly monitoring cardiac hemodynamics of a mammal subject, comprising:
implanting a bio-sensing module in a heart or an artery of the mammal subject, wherein the bio-sensing module is configured to continuously monitor cardiac functions of the mammal subject;
implanting a wireless electronic subsystem within a skin of the mammal subject, and electrically connecting the wireless electronic subsystem to the bio-sensing module through insulated flexible wires; and
wirelessly communicating the wireless electronic subsystem an external wireless power transfer (WPT) module and an external user interface module, wherein the wireless electronic subsystem is configured to:
wirelessly receive power transferred from the external WPT module, and provide the power to the bio-sensing module; and
obtain sensing signals of the cardiac functions monitored by the bio-sensing module, and wirelessly transmit the sensing signals obtained to the external user interface module.

44. The method of claim 43, wherein the wireless electronic subsystem is disposed between a fat layer and a dermis layer of the skin of the mammal subject.

45. The method of claim 43, wherein the wireless electronic subsystem is wirelessly communicated to the external WPT module and the external user interface module bio-sensing module under a Bluetooth low energy (BLE) communication protocol.

46. The method of claim 43, wherein the external WPT module and the external user interface module collectively form a wearable external monitoring device.

47. The method of claim 43, wherein the bio-sensing module has a multilayered structure comprising:
a substrate;
a plurality of sensors disposed on the substrate, configured to measure bi-directional flow rate, pressure and temperature of blood of the mammal subject;
a plurality of flexible and stretchable interconnects electrically connecting the sensors; and
an elastomeric encapsulation layer at least partially surrounding the substrate, the sensors and the flexible and stretchable interconnects.

48. The method of claim 47, wherein each of the sensors is formed by a piezoelectric monocrystalline silicon nanomembrane (Si-NM) strain gauge.

49. The method of claim 48, wherein the sensors comprise:
a flow sensor configured to measure the bi-directional flow rates of the blood;
a pressure sensor configured to measure the pressure of the blood; and
a temperature sensor configured to measure the temperature of the blood.

50. The method of claim 49, wherein the flow sensor is formed as a three-dimensional fin structure by the Si-NM strain gauge to measure the bi-directional flow of the blood based on tensile and compressive forces to the Si-NM strain gauge caused by the bi-directional flow rate of the blood.

51. The method of claim 50, wherein a relationship between a strain ε measured by the Si-NM strain gauge to bi-directional flow velocity v of the blood is:
$$ε ( \% ) = {c}_{0} \cdot {v}^{2} + {c}_{1} \cdot v ,$$
wherein c₀ and c₁ are constant coefficients.

52. The method of claim 51, wherein for the bi-directional flows of the blood, values of c₀ and c₁ are:
for a forward flow of the blood, c_{0,f}=0.048 and c_{1,f}=0.0047; and
for a back flow of the blood, c_{0,b}=−0.0497 and c_{1,b}=0.0049.

53. The method of claim 49, wherein the pressure sensor is formed by disposing the Si-NM strain gauge on an air-filled cavity on the substrate to be compressed by the pressure of the blood.

54. The method of claim 53, wherein a relationship between a strain ε measured by the Si-NM strain gauge to bi-directional flow velocity v of the blood is:
$$ε ( \% ) = c \cdot {[ Δ P \sqrt{S} / {\tilde{E}}_{S i {O}_{2}} {h}_{S i {O}_{2}} + {\tilde{E}}_{S i} {h}_{S i} + {\tilde{E}}_{P I} {h}_{PI} ]}^{\frac{2}{3}} = α \cdot Δ {P}^{\frac{2}{3}} ,$$
wherein c is a constant, S is the surface area, ΔP is the pressure, E is a Young's modulus of the Si-NM strain gauge, and h is a thickness of the Si-NM strain gauge.

55. The method of claim 43, wherein the wireless electronic subsystem comprises:
a plurality of electronic components;
a plurality of flexible antenna coils electrically interconnected to the electronic components; and
a plurality of bio-compatible encapsulation layers encapsulating the electronic components and the flexible antenna coil.

56. The method of claim 55, wherein the flexible antenna coils comprises a receiving coil and a transmitting coil, and the electronic components comprise:
a power management module electrically connected to the receiving coil, configured to receive and convert the power wirelessly received by the receiving coil, and to provide the power to the bio-sensing module;
a Bluetooth low energy (BLE) system on a chip (SoC) electrically connected to the power management module, configured to receive and transmit the power converted by the power management module, and to control the transmitting coil to wirelessly transmit the sensing signals to the external user interface module; and
analog front-end (AFE) circuits electrically connected to the BLE SoC, configured to transmit the power received from the BLE SoC, to obtain analog signals from the bio-sensing module as the sensing signals of the cardiac functions, and to transmit the sensing signals to the BLE SoC.

57. The method of claim 56, wherein the power management module comprises:
a bridge rectifier electrically connected to the receiving coil to perform full-wave rectification to the power received by the receiving coil;
a charge pump converter electrically connected to the bridge rectifier, configured to regulate voltage of the power received by the receiving coil; and
a pair of supercapacitors (SCs) electrically connected to the charge pump converter, configured to be controlled by the voltage regulated by the charge pump converter to perform a short-term energy buffer during periods with an angular mismatch between the receiving coil and the transmitting coil.

58. The method of claim 57, wherein the BLE SoC comprises:
a plurality of analog to digital converters (ADCs) configured to convert analog signals received by the AFE circuits to the digital data;
a general-purpose input/output (GPIO) pin configured to supply the regulated voltage to the AFE circuits; and
a central processing unit (CPU) configured to receive the regulated voltage of the power from the power management module, to provide the regulated voltage to the GPIO pin, to obtain the digital data from the ADCs, and to control the transmitting coil to wirelessly transmit the sensing signals to the external user interface module based on digital data obtained.

59. The method of claim 56, wherein the external WPT module comprises a main transmitting coil in a rectangular shape and a secondary transmitting coil in a circular shape, wherein the main transmitting coil is located at a center of the secondary transmitting coil to wirelessly transfer the power to the receiving coil of the wireless electronic subsystem, and the secondary transmitting coil increases a working range of the receiving coil of the wireless electronic subsystem.
